(19) United States
(12) Patent Application Publication
(10) Pub. No.: US 2022/0249001 A1
(43) Pub. Date: Aug. 11, 2022

(54) PATIENT MONITORING DEVICE

(71) Applicant: Murata Vios, Inc., Woodbury, MN (US)

(72) Inventors: Atsuhiko HIRATA, Woodbury, MN (US); Rachel MYERS, Woodbury, MN (US); Nobuhiro KONDO, Woodbury, MN (US)

(21) Appl. No.: 17/668,709

(22) Filed: Feb. 10, 2022

Related U.S. Application Data
(60) Provisional application No. 63/148,296, filed on Feb. 11, 2021.

(51) Int. Cl. A61B 5/274; A61B 5/256; A61B 5/257; A61B 5/00; A61B 5/024; A61B 5/021; A61B 5/145; A61B 5/01; A61B 5/08; A61B 5/11

(52) U.S. Cl. CPC A61B 5/274; A61B 2562/166; A61B 5/257; A61B 5/6823; A61B 5/024; A61B 5/021; A61B 5/14532; A61B 5/01; A61B 5/0816; A61B 5/14542; A61B 5/4875; A61B 5/1118; A61B 5/1116; A61B 2560/0214; A61B 5/256

(57) ABSTRACT

A patient-monitoring device includes a housing including a first opening, a snap located in the first opening to connect to an electrode through the first opening, and a dielectric potting compound covering the snap. A method of manufacturing a patient-monitoring device includes attaching a wire to a snap, providing a housing, providing a substrate including a connector and circuitry components, installing the snap into the housing, installing the substrate into the housing, providing a dielectric potting compound into the housing such that the dielectric potting compound covers the snap and the substrate, and curing the dielectric potting compound.

### CROSS REFERENCE TO RELATED APPLICATIONS

This application claims benefit to U.S. Patent Application No. 63/148,296, filed on Feb. 11, 2021, the entire contents of which are hereby incorporated herein by reference.

### BACKGROUND OF THE INVENTION

### 1. Field of the Invention

The present invention relates to a patient-worn monitoring device that is temporarily affixed or adhered to a patient's skin.

### 2. Description of the Related Art

Many different types of patient monitoring systems require a direct electrical interface to the skin of a patient. In some applications, the direct electrical interface to the patient's skin is to sense electrical signals present at that skin location; while in other applications, the direct electrical interface is to apply an electrical current stimulation signal at that skin location. Therefore, the patient monitoring systems typically require a patient-worn sensor assembly that detects, records, and communicates patient data. As such, the patient-worn sensor assembly can include several structural features that can provide increased signal quality, reduction in signal noise, increased patient comfort, increased reliability, and increased adhesion to a patient's skin.

The patient-worn sensor can receive vital-sign information, such as blood pressure, body temperature, respiratory rate, blood oxygenation, electrocardiogram (ECG), heart rhythm, heart rate, blood glucose level, and hydration (bio-impedance) levels, etc. The patient-worn sensor can also track and record additional information about patients, including patient movement, activity, and sleep patterns.

A conventional patient-worn sensor collects information sensed at the patient's skin and wirelessly transmits the data to another device of a monitoring system (e.g., bed-side monitor, tablet device, mobile phone, central processing server, etc.), which in turn can be connected to a network system of a hospital, clinic, or home-based monitoring system. Such a patient-worn sensor can include an adhesive electrode assembly with multiple individual electrodes that are attached to the patient's skin, and a sensor assembly that includes all of the sensing, processing and communication electronics, and a power supply in a self-contained sensor-transmitter device. In this conventional patient-worn sensor, the electrode assembly provides a direct electrical interface, an adhesive to attach to the patient's skin, and a platform to which the sensor assembly connects and is supported by.

FIG. 1 shows a conventional patient-worn sensor of an ECG monitoring system that includes an adapter-sensor assembly 1000 with separate adapter 1110 and sensor 1120 and several leads for connecting to a patient's skin. The two-piece adapter-sensor assembly 1000 includes the sensor 1120 attached to the adapter 1110. The adapter 1110 can be attached to a patient's skin by tacky monitoring electrodes. As shown in FIG. 1, the leads can include, for example, a Mod lead to sense respiration sensing and ECG leads such as an RA lead, an LL lead, and a V lead. The leads are located at the end of cables connected to the adapter-sensor assembly 1000. The leads snap onto electrodes that are adhered to the patient's skin. As discussed below, the adapter-sensor assembly 1000 includes snaps 1111 on the rear that attach to electrodes. As shown, the patient-worn sensor can also include a pulse oximeter (ox) sensor 1131 that can be clipped onto the patient's finger to measure oxygen saturation level. FIG. 2 shows that the adapter-sensor assembly 1000 can be attached to a patient's chest area 1180 with cables 1140 connected to the leads that are attached to patient's right-hand side and to an area near the patient's waist. The cable 1130 for the pulse ox sensor 1131 is attached to the patient's left-hand side. Conventional patient-worn sensors are cumbersome because pulse ox sensor cables 1130 are only attached to the left side of a patient-worn sensor, for example, as shown in FIG. 2. Accordingly, routing the pulse ox sensor 1131 to a patient's right hand in the configuration shown in FIG. 2 would require inconvenient routing of the cable 1130 over or around the adapter-sensor assembly 1000 creating unnecessary excess slack in the wiring.

All patient-worn sensors should be comfortable for the patient. Additionally, the components should be flexible, dimensionally small, chemically inert, resistant to disinfectants, nontoxic, hypo-allergenic to the human body, easy to use, rugged enough to survive impacts from drops, and provide one or more methods for attaching to the patient.

Conventional patient-worn sensors can have problems with adhesion and electrical contact. That is, patient-worn sensors need to be reliable and maintain contact with the patient throughout all standard use conditions, including, for example, during patient movement. Furthermore, patient-worn sensors need to minimize variations across electrodes caused by patient movement that induces mechanical stress on the electrodes or patient contact points.

FIG. 3 is a perspective view of the conventional adapter 1110, and FIG. 4 is a perspective view of the conventional sensor 1120. The adapter 1110 can include a push tab 1114, a socket connector (hidden in the view but located near the push tab 1114), arms 1116, and an area in which the cables of leads 1140 and a cable 1130 of the pulse ox sensor 1131 are attached. The socket connector receives a mating electrical connector 1123 on the sensor 1120. The push tab 1114 on the adapter 1110 is used with a push tab 1124 on the sensor 1120 to join and disconnect the sensor 1120 from the adapter 1110. The arms 1116 are located on opposing sides of the adapter 1110 to align and secure the sensor 1120.

As shown in FIG. 4, the sensor 1120 can include the electrical connector 1123 and the push tab 1124. The electrical connector 1123 plugs into the socket connector of the adapter 1110 and is used to transmit and receive power and electrical signals between the sensor 1120 and the adapter 1110. As shown, the sensor 1120 also includes a receptacle 1125 that can be used to charge a battery (not shown) in the sensor 1120 or to transmit/receive data when the sensor 1120 is not connected to the adapter 1110.

FIG. 5 shows how the sensor 1120 can be aligned by the arms 1116 of the adapter 1110 and moved in the direction of the arrow to engage the connector 1123 into the socket connector on the adapter 1110. As shown in FIG. 5, the sensor 1120 slides into the adapter 1110 from above, which requires the pulse-ox cable 1130 to be located on the side of the adapter 1110 to avoid interference with the sensor 1120 as the adapter 1110 is slid into the adapter 1110. Because the pulse-ox cable 1130 is located on the side of the adapter 1110, the sensor 1120 can only be used on the patient's left arm. FIG. 6 shows the adapter-sensor assembly 1000 with the sensor 1120 in place and fully engaged with the adapter 1110.

FIG. 7 is a rear view of the adapter-sensor assembly 1000 that shows two snaps 1111-RL and 1111-LA surrounded by soft boots 1119 embedded in the rear of the adapter 1110 that connect, respectively, to electrodes for the RL and LA electrodes. FIGS. 8A to 8C show conventional skin electrodes 1141 mounted on a patient's chest and a conventional approach to avoid a pop-off problem that can occur when the patient's skin stretches during movement, causing relative movement between the conventional skin electrodes.

A conventional skin electrode assembly is connected by an electrical lead and a corresponding cable of the monitoring system. In this conventional skin electrode assembly, the patient-worn assembly typically includes an electrode assembly with one or more electrodes that can be adhered to the patient's skin and an associated connector assembly with an associated number of contacts of the one or more electrodes of the electrode assembly. The connector assembly can be connected to a monitoring-system lead to provide an electrical connection between the one or more electrodes of the electrode assembly and the sensing-and-processing circuitry of the monitoring system.

FIG. 8A shows two adjacent electrodes 1141 mounted on a patient's chest and arranged to attach to the snaps 1111 embedded in the rear of the conventional adapter 1110 as shown in FIG. 7. When the adapter 1110 is snapped onto the skin-mounted electrodes 1141, the soft boots 1119 absorb motion caused by flexing with the patient's skin and can absorb shock due to motion caused by other movements of the patient, such as walking, running, rolling over, etc. Additionally, the soft boots 1119 also absorb instantaneous mechanical shock to the snaps 1111 and to an internally coupled trace caused by snapping and unsnapping a mating snap connector of the mounted electrodes 1141. The soft boots 1119 can be made using an over-molding process from a material having less rigidity than a material used to form other portions of the adapter housing, such that the soft boots 1119 are integrated with a portion of a housing of the adapter 1110 as an integral piece.

Once attached to the patient, the patient is free to move, which can cause shifting in the relative location of contact points on the patient's skin at which the electrodes 1141, which are attached to adhesive electrode pads, contact the patient. However, as shown in FIG. 8B, the skin over the patient's chest can stretch when the patient moves their arms or shoulders, causing the two adjacent electrodes 1141 to separate horizontally. As shown in FIG. 8C, the soft boots 1119 can flex and absorb some or all of the motion between the skin-mounted Electrode and the adapter-sensor assembly 1000 caused by movement of the patient, thereby reducing negative effects on signal integrity and quality received by the electrodes 1141 of the adhesive electrode pad.

The conventional arrangement described above can cause the snaps 1111 on the rear of the adapter 1110 to disconnect from the skin-mounted electrodes 1141. That is, certain skin electrodes tend to pop off from the snaps 1111 of the adapter 1119 because of interference between a soft boot 1119 and a mating snap 1142 on a skin-mounted electrode 1141. The snap 1111 inside the adapter 1110 is usually female and the mating snap 1142 on an electrode 1141 is usually male. Furthermore, if the diameter of the hole on the soft boot 1119 is increased to prevent such interference and consequent pop-off, the snaps 1111 of the adapter 1110 can pull out of the soft boot 1119 when removing the adapter 1110 from skin-mounted electrodes 1141. A detailed explanation of the pop-off and pull-out problems is described with respect to FIGS. 9A to 12.

FIGS. 9A and 9B show two conventional skin electrode configurations. The configuration shown in FIG. 9A has a metal snap 1142A, and the configuration shown in FIG. 9B has a plastic snap 1142B. An example of a skin electrode with a metal snap is the 3M RED DOT 2560, and an example of the skin electrode with a plastic snap is the 3M RED DOT 2570. The skin electrodes with plastic snaps 1142B can be radiolucent, that is, transparent to X-rays. FIG. 10A shows a top view of the metal snap 1142A on the left and the plastic snap 1142B on the right, and FIG. 10B shows a side view of the metal snap 1142A on the left and the plastic snap 1142B on the right. As shown in FIGS. 10A and 10B, the geometries of the metal snap 1142A and the plastic snap 1142B are different in that the diameter and thickness of the plastic snap 1142B are larger than the diameter and thickness of the metal snap 1142A. Although the soft boot 1119 can work well with the metal snap 1142A, the hole in the conventional soft boot 1119 is too small to allow the snaps 1111 of the adapter 1110 to properly engage with the plastic snap 1142B. Accordingly, a skin-mounted electrode 1141 with a plastic snap 1142B is able to easily pop off and to disconnect from the adapter 1110.

The hole size in the soft boot 1119 can be increased as shown in FIG. 11B where the diameter of the holes in the soft boots 1119′ in the adapter 1110′ are larger than those of the soft boots 1119 provided in the conventional adapter 1110 shown in FIG. 11A. Although increasing the hole size reduced the occurrence of the plastic snaps 1142B from popping off of the soft boots 1119′, the flexible soft boots 1119′ with the larger holes were no longer strong enough to keep the snap contained within the soft boots 1119′ in some circumstances. That is, the snaps 1111 tended to pull out from the soft boots 1119′ with larger holes when disengaging the adapter 1110 from the skin-mounted electrodes 1141. An example of this failure is shown in FIG. 12, where even with the original hole diameter in the soft boots 1119 shown in FIG. 11A, pull-out can occur (indicated by the arrow) if an unusually high pull force is applied to the snap 1111 of the adapter 1110.

FIGS. 13 and 14 are views of the adapter 1110 with the top cover removed. As shown, the adapter 1110 includes the connector 1123 (for connecting to the sensor 1120) mounted on a rigid printed circuit board (PCB) 1121. The rigid PCB 1121 is connected to a flex (flexible) PCB 1122 via a pair of mating stacking connectors 1126 shown in FIG. 14. Although not shown, the flex PCB 1122 includes wiring and electronic components mounted to the flex PCB 1122. The flex PCB 1122 can be partially reinforced with stiffeners. The wiring includes interconnection traces connected to soldering pads 1127 where ends of wires for the leads can be directly soldered or in which connectors for the leads are soldered. The wiring also includes interconnections to the electrode snaps 1111 that are retained by the soft boots 1119.

For when the patient requires defibrillation while attached to the patient monitoring device, high voltage resistors 1128 are located in line between each electrode 1141 and the main circuitry 1129 of the adapter-sensor assembly 1000 (other than the RL lead which is the common electrode for right leg drive) to reduce current from the high voltage applied during defibrillation. The high voltage resistors 1128 (which are not shown in FIGS. 13 and 14 but are shown in FIG. 15) are mounted on the flex PCB 1122, and are encapsulated with a dielectric material, such as glue (not shown), including the side facing the flex PCB 1122. The soldering pads 1127 of the wires are also covered by a dielectric material, such as glue, to prevent voltage arcing between nearby soldering pads 1127 or circuit components. However, the snaps 1111 are not covered by a dielectric material, such as glue, to allow movement of the snaps 1111 and the soft boots 1119. To minimize chances for voltage arcing between (a) the snaps 1111 and the line between each of the snaps 1111 and the corresponding high voltage resistor 1128 and (b) other uncovered conductor surfaces, the design must include sufficient distance between (a) the snaps 1111 and the line between each of the snaps 1111 and the corresponding high voltage resistor 1128 and (b) other uncovered conductor surfaces. Thus, as shown in FIG. 15, the high-voltage resistor 1128 is provided between the electrode 1141 and the main circuit 1129, and conductor surfaces in an area 1150 must be covered by an insulator or be sufficiently distanced from each other. Because of the risk of voltage arcing, extra space is required around each snap 1111, which makes the adapter 1110 larger than if the adapter 1110 did not need the extra space required for the high-voltage resistor 1128.

### SUMMARY OF THE INVENTION

To overcome the problems described above, preferred embodiments of the present invention provide patient-monitoring devices that achieve significantly reduced weight and size, while protecting internal circuitry from high voltages and electrical arcing during defibrillation by using a potting compound to cover the internal circuitry.

According to a preferred embodiment of the present invention, a patient-monitoring device includes a housing including a first opening, a snap located in the first opening to connect to an electrode through the first opening, and a dielectric potting compound covering the snap.

The patient-monitoring device can further include a flat surface surrounding the first opening. The patient-monitoring device can further include a protrusion that defines the flat surface.

The housing can further include a plurality of ribs spaced around the first opening. The patient-monitoring device can further include a second opening, where the first opening and the second opening are aligned to be vertical or substantially vertical when the patient-monitoring device is mounted to a patient. The patient-monitoring device can further include a first cavity that defines the first opening and a second cavity that defines the second opening.

The patient-monitoring device can further include a battery pack that is attached and detached from the housing, and the battery pack can include a battery to power the patient-monitoring device. The housing can include a first connector, and the housing can align and restrict a direction in which the battery pack is attached and detached to the first connector. The housing can include a first lock, and the battery pack can include a second lock that communicates with the first lock to lock the battery pack to the housing. The battery pack can include a second connector that mates with the first connector, and the battery pack can include a structure to prevent insertion of an incompatible connector.

The housing can include a structure to prevent insertion of an incompatible connector. The battery pack can be attached and detached from a side of the housing. A portion of the housing may not be overlapped, in plan view, with the battery pack when the battery pack is attached to the housing.

The patient-monitoring device can further include an antenna within the portion of the housing not overlapped, in plan view, with the battery pack when the battery pack is attached to the housing. The dielectric potting compound may not cover the antenna. A relative permittivity of the dielectric potting compound can be less than 4. The housing can further include an antenna, and the dielectric potting compound may not cover the antenna.

The patient-monitoring device can further include a printed circuit board that can be located in the housing. The printed circuit board can include holes. The dielectric potting compound can cover the printed circuit board. The patient-monitoring device can further include a secondary printed circuit board that is in the housing, and the dielectric potting compound can be provided not to cover the secondary printed circuit board. The housing can include a retaining wall that extends around a perimeter of the printed circuit board, and the dielectric potting compound can be within the retaining wall. At least a portion of the housing can be transparent. At least one of a top surface, a bottom surface, a left surface, a right surface, a front surface, and a back surface of the battery pack may not oppose the housing. The housing can include openings that can be an identical shape and that can each receive a cable or plug.

According to a preferred embodiment of the present invention, a method of manufacturing a patient-monitoring device includes attaching a wire to a snap, providing a housing, providing a substrate including a connector and circuitry components, installing the snap into the housing, installing the substrate into the housing, providing a dielectric potting compound into the housing such that the dielectric potting compound covers the snap and the substrate, and curing the dielectric potting compound.

In the method, the housing can include an opening, the snap can be located adjacent to the opening, the opening can be covered prior to the providing the dielectric potting compound, and the dielectric potting compound can cover the snap without flowing through the opening. In the method, a relative permittivity of the dielectric potting compound can be less than 4. The method can further include providing a secondary substrate and installing the secondary substrate into the housing, and the dielectric potting compound can be provided to not cover the secondary substrate. The housing can include a retaining wall that limits the potting compound to an area surrounding the substrate.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 shows a conventional patient-worn sensor of an ECG monitoring system.

FIG. 2 shows a conventional patient-worn sensor in contact with a patient's skin.

FIG. 3 shows a conventional adapter.

FIG. 4 shows a conventional sensor.

FIGS. 5 and 6 show a conventional adapter-sensor assembly.

FIG. 7 is a rear view of a conventional adapter.

FIGS. 8A to 10B show conventional skin-mounted electrodes.

FIGS. 11A, 11B, and 12 show problems with conventional adapters.

FIGS. 13 and 14 show interior components of a conventional adapter.

FIG. 15 shows a conventional protection circuit.

FIGS. 16 and 17 show a patient monitoring device according to a preferred embodiment of the present invention.

FIGS. 18-20 show a chest device according to a preferred embodiment of the present invention.

FIG. 21-23 show a battery pack according to a preferred embodiment of the present invention.

FIGS. 24-26 show connection features of the battery pack and the chest device according to a preferred embodiment of the present invention.

FIGS. 27-31B show the orientation of the patient monitoring device on a patient according to a preferred embodiment of the present invention.

FIGS. 32 and 33 are views of a bottom housing of the chest device according to a preferred embodiment of the present invention.

FIGS. 34-44 show an assembly process of the chest device according to a preferred embodiment of the present invention.

FIGS. 45 and 46 show an antenna implementation according to a preferred embodiment of the present invention.

FIGS. 47A to 47C show horizontal motion but not vertical motion of skin electrodes when a patient moves.

FIGS. 48-51 show applying potting compound to the housing of the chest device.

FIG. 52 shows a chest device, an updating cable, and a mobile device.

FIG. 53 shows a charging station for the battery packs.

FIGS. 54-57 show a modified chest device according to a preferred embodiment of the present invention.

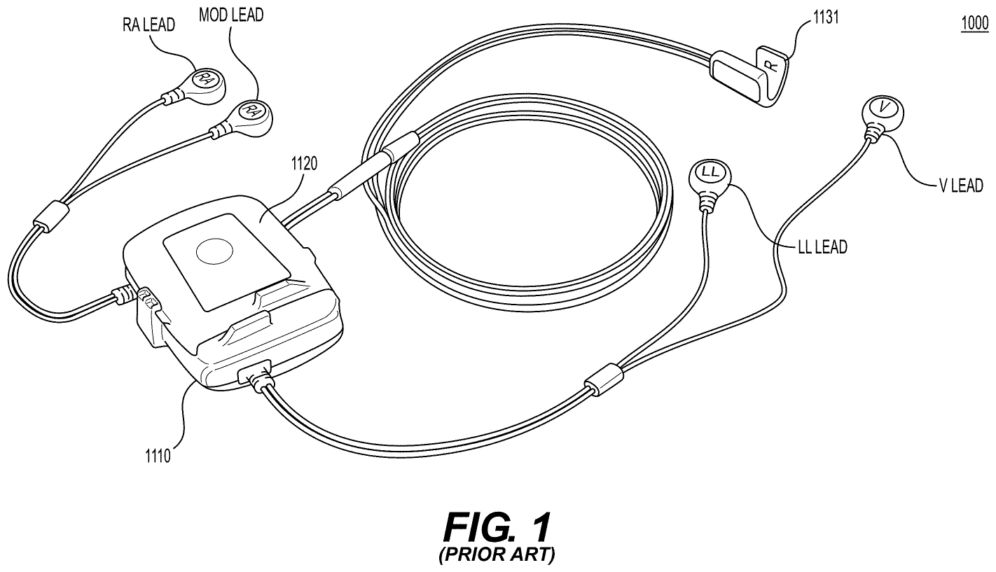

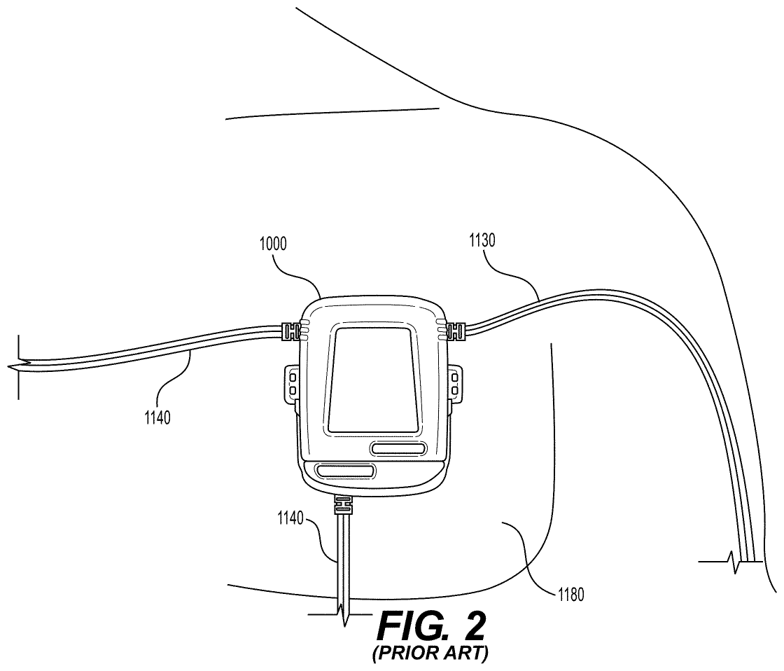

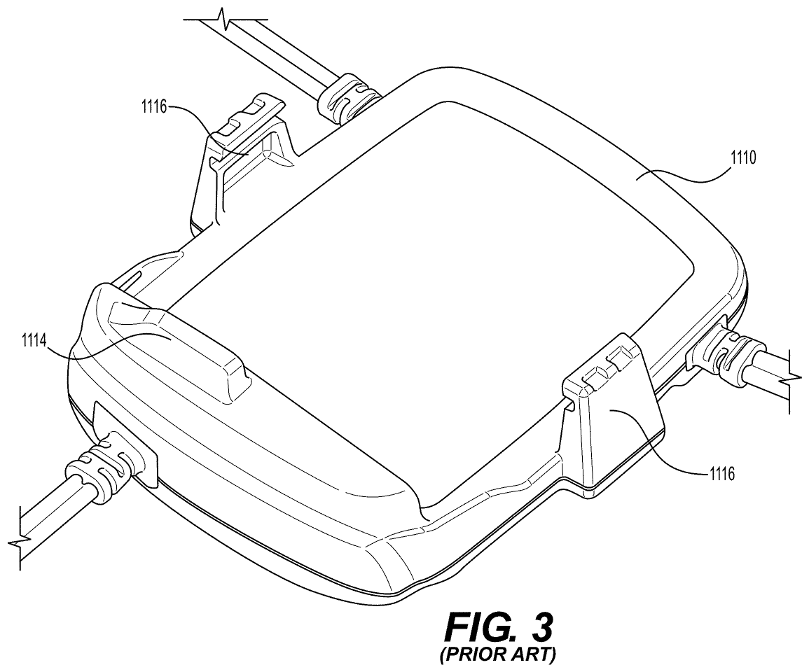

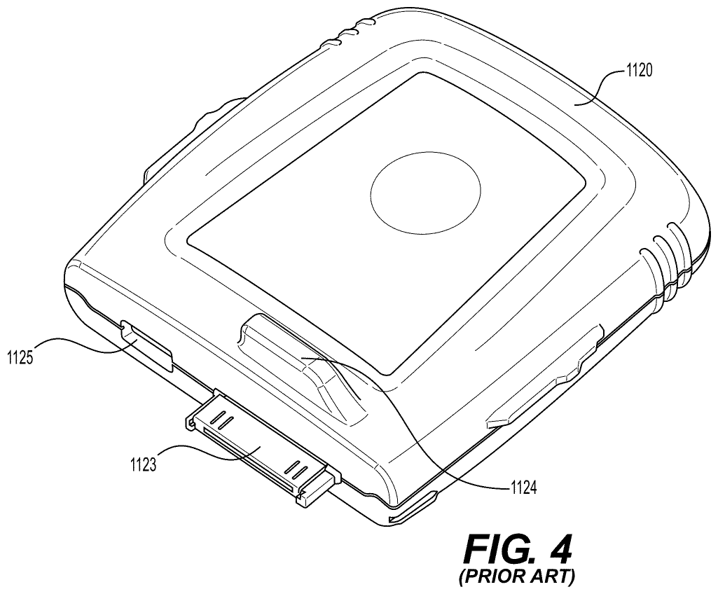

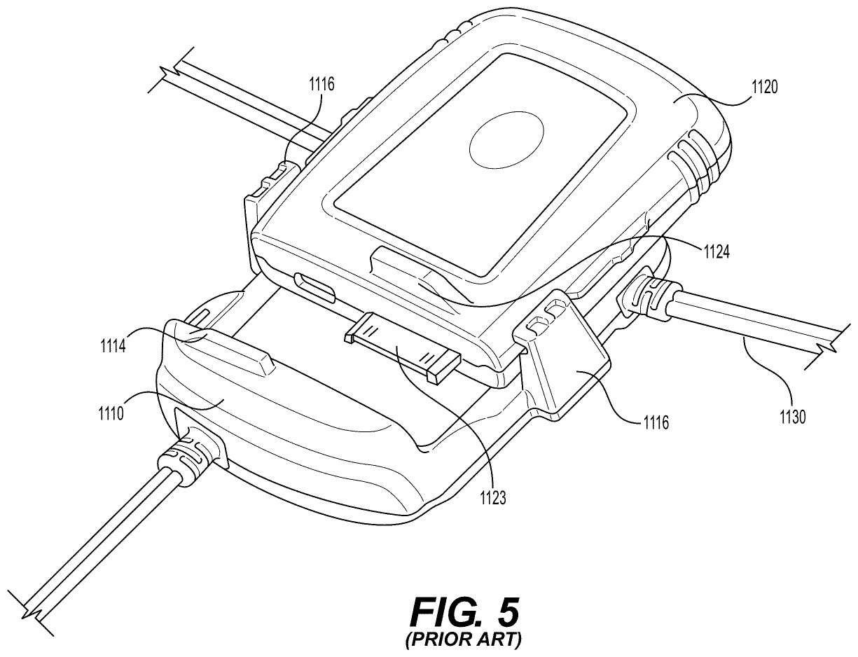

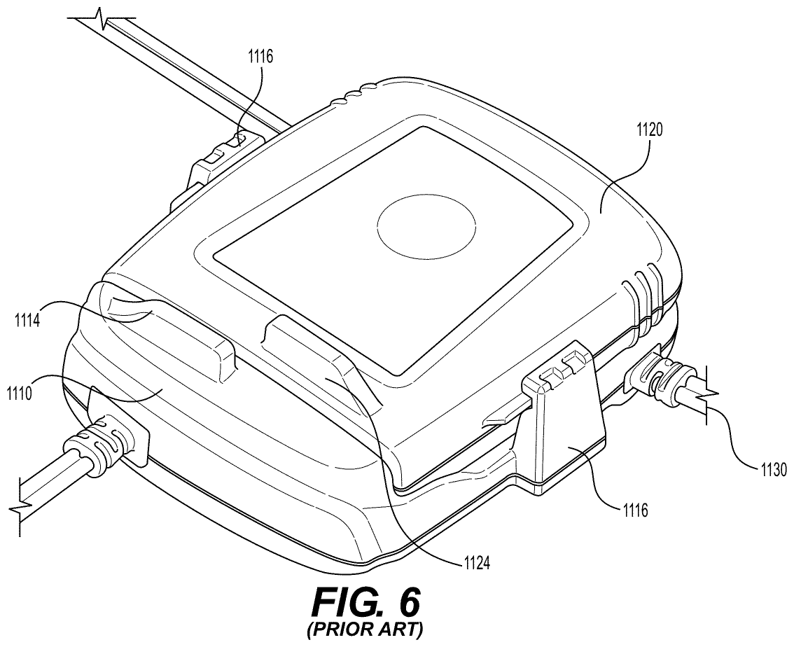

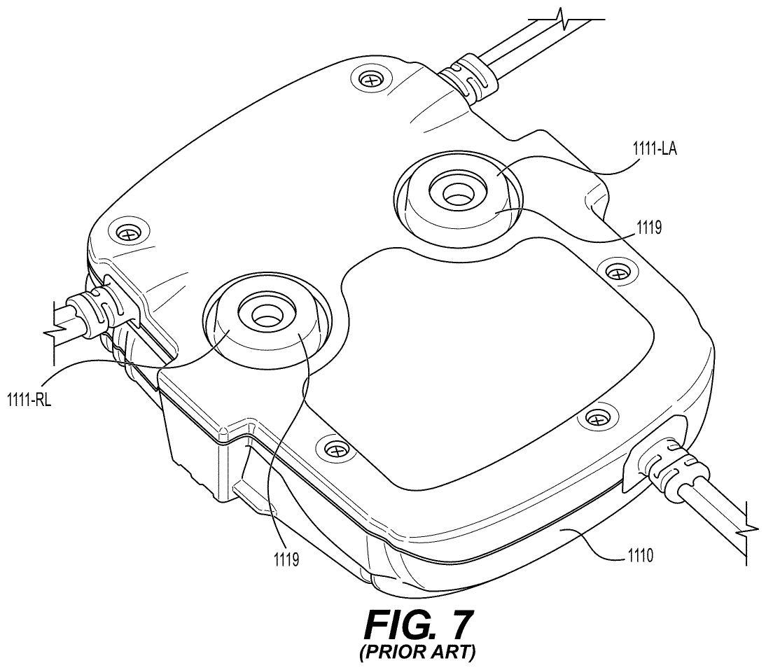

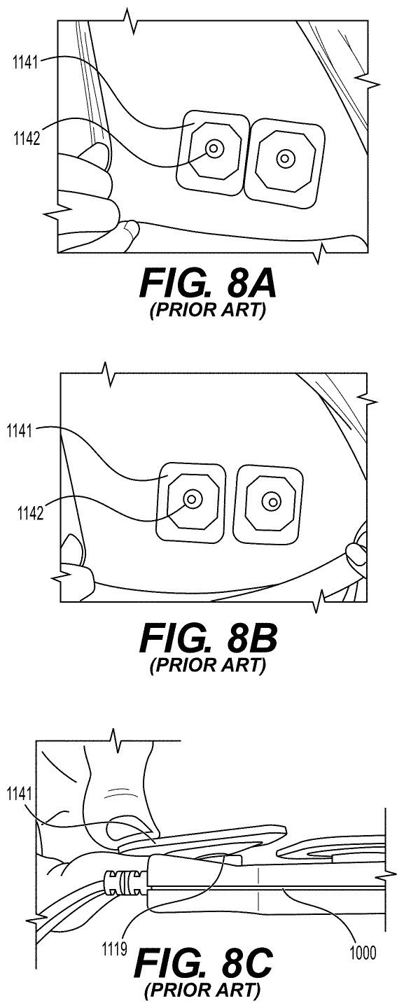

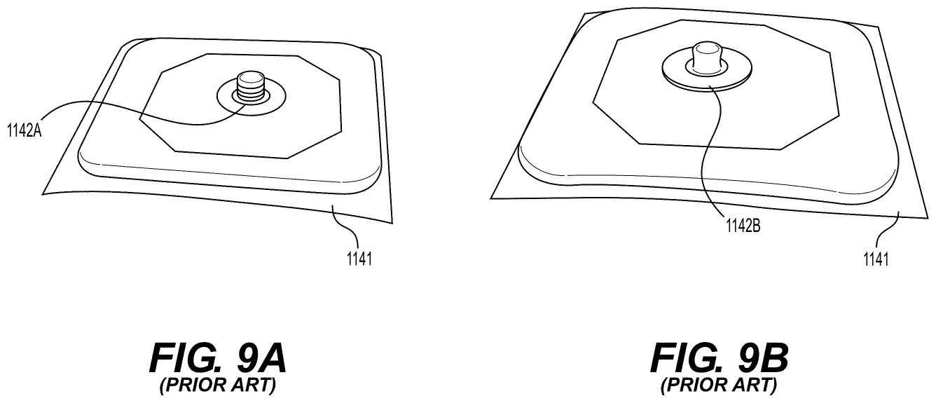

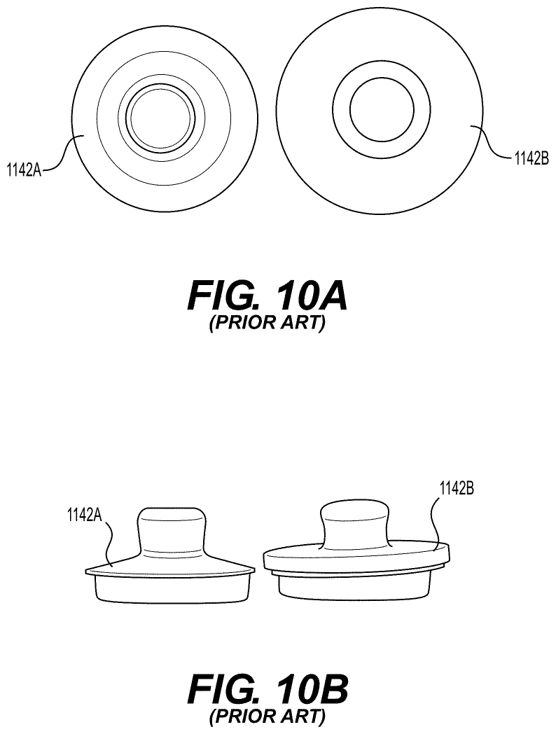

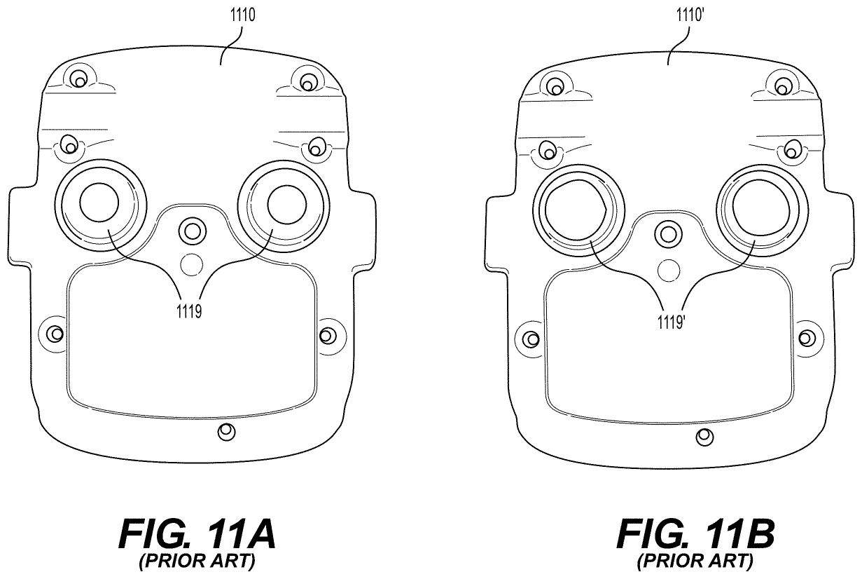

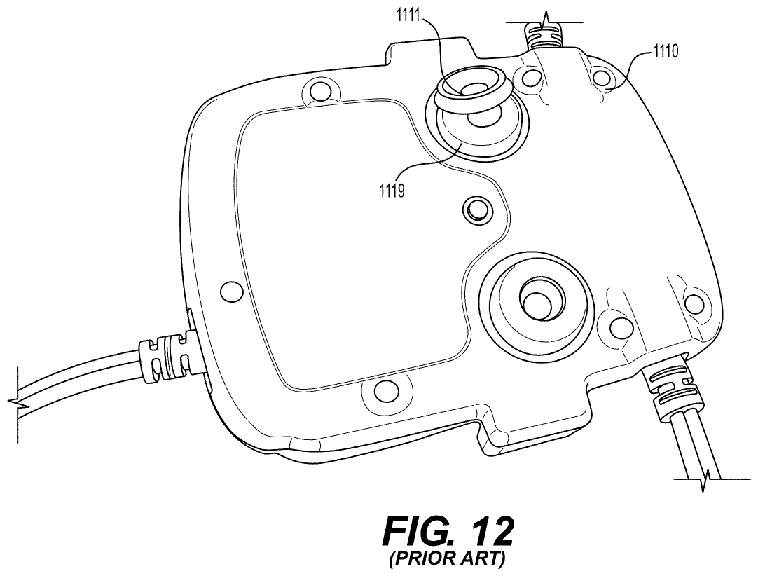

### DETAILED DESCRIPTION OF PREFERRED EMBODIMENTS

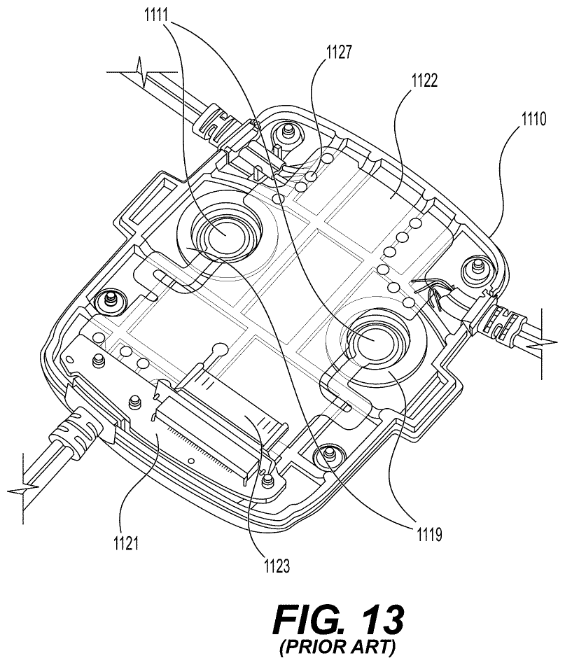

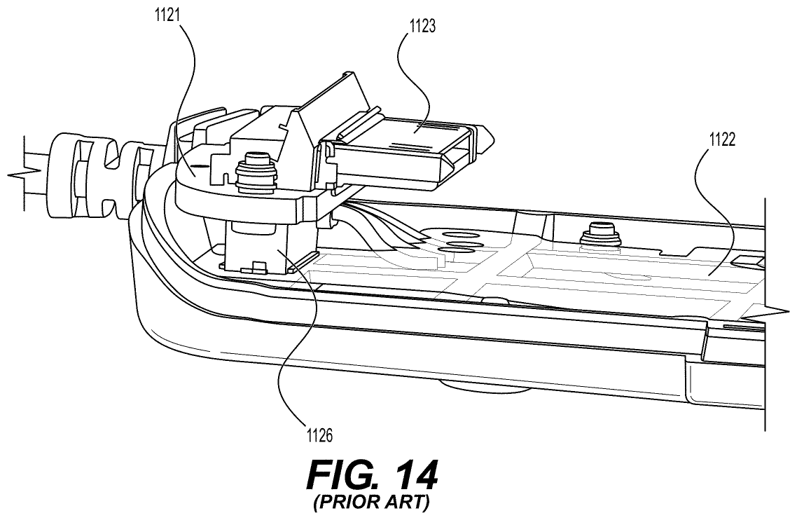

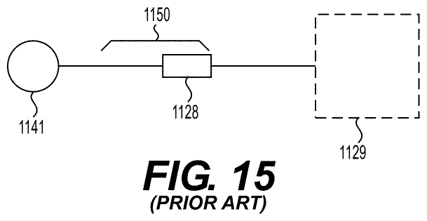

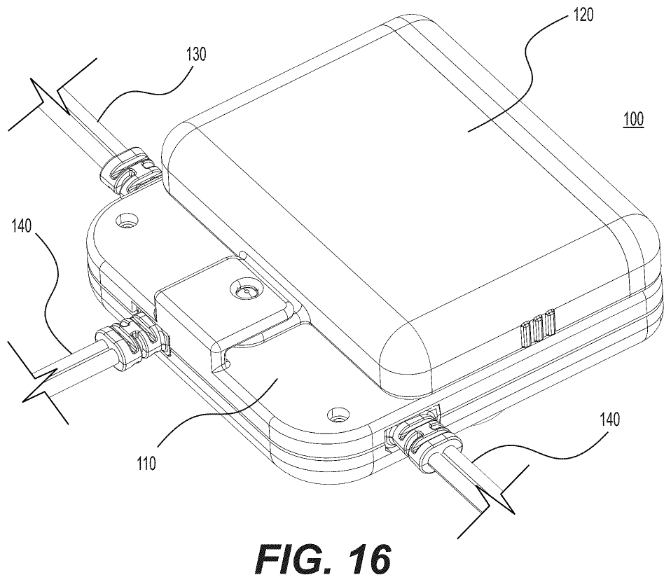

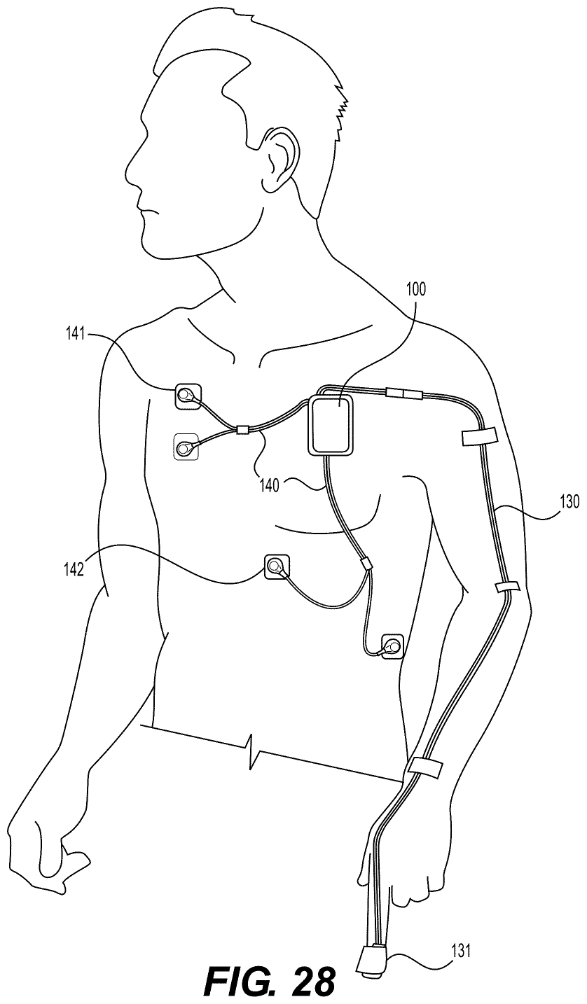

FIG. 16 shows a patient monitoring device 100 according to a preferred embodiment of the present invention. In the example shown in FIG. 16, the chest device 110 of the patient monitoring device 100 can be attached to the chest of a patient. However, the patient monitoring device 100 can be attached to one or more other areas on the patient's body. The patient monitoring device 100 can detect, record, store, and transmit various vital signs and other information of the patient. For example, the patient monitoring device 100 can include several adherent electrodes (see, for example, FIG. 28) that contact the patient's skin to measure various biological information, vital signs, and patient information including, but not limited to, heart rhythm, heart rate, blood pressure, body temperature, respiratory rate, blood oxygenation, blood glucose level, hydration levels, perspiration, and bio-impedance. The patient monitoring device 100 can also track patient motion, movement, activity, position, posture, and physical location.

The patient monitoring device 100 can also communicate with one or more other computing devices, either through wired or wireless communication. For example, the patient monitoring device 100 can use Bluetooth, Wi-Fi, or a cellular communication protocol to communicate with other computing devices such as bedside monitors, personal computers, tablet devices, mobile phones, central servers, or a cloud-based network. As an example, the patient monitoring device 100 can transmit vital-sign information collected from the patient to a tablet device or a personal computer that operates as a bedside monitor. The tablet or personal computer can process received information and display the information in a readily understandable format to a caretaker or other user. For example, a tablet device can receive vital-sign information from the patient monitoring device 100 through a Bluetooth connection and display an electrocardiogram (ECG) waveform of the patient, as well as information on the patient's heart rate, respiration rate, blood oxygenation level, body temperature, and/or other vital signs. As another example, the patient monitoring device 100 can be periodically connected to a computing device (such as a bedside monitor) through a wired connection to allow information collected by the patient monitoring device 100 to be stored, processed, and displayed by the computing device and/or transferred to one or more other computing devices (e.g., personal computers, servers located at the hospital, cloud storage servers, etc.).

Furthermore, information recorded by the patient monitoring device 100 can be transmitted to other computing devices to provide real-time or near real-time analysis of the patient's condition, and to provide tracking of vital-sign information of the patient over time. For example, the information recorded by the patient monitoring device 100 can be transmitted to a display device to allow caregivers to observe the information and adjust patient care based on the information. The information can also be transmitted to a central information repository to log and store historical vital-sign and other information of the patient. Both real-time and historical vital-sign information, and other information, of a patient can be accessed by caregivers who are not at the same physical location as the patient. For example, vital-sign information collected by the patient monitoring device 100 can be transmitted to a mobile device owned by the patient (e.g., a smart phone) to allow the patient to view the information. The information can further be transmitted to a central server that can be accessed by one or more caregivers (e.g., using personal computers or mobile devices) to allow the caregivers to view the collected information and make patient care decisions for the patient from a location that is remote from where the patient is located.

Other components that can be included as part of the patient monitoring device 100 include a power supply, buttons, or other input mechanisms for receiving user input, one or more audible alarms or speakers, and lights or a display screen. The patient monitoring device 100 can further include input mechanisms such as, for example, buttons, keys, or a touch screen. The input mechanisms can allow the patient or a caregiver to adjust settings for the patient monitoring device 100, perform various tests (for example, sensor tests, battery power level tests, etc.), or reset one or more alarms for the patient monitoring device 100. The input mechanisms can also allow the patient to place a distress call (e.g., to a caregiver or to a hospital alert system) if the patient needs assistance.

As shown in FIG. 16, the patient monitoring device 100 can include a chest device 110 to mount to the patient, a battery pack 120 to provide power the chest device 110, a pulse ox cable 130 contacting one side of the chest device 110, and two cables 140 each connected to different sides of the chest device 110 that attach signal leads to the chest device 110. The cables 140 can be ECG cables. The battery pack 120 can include standard disposable batteries or a rechargeable battery and is removable such that it can be replaced with a different battery pack.

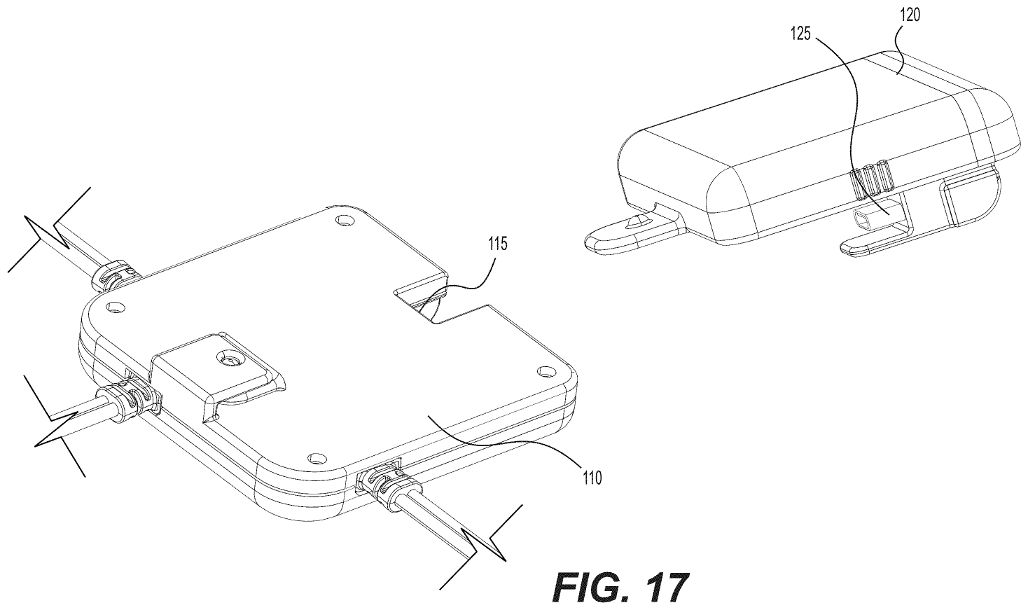

FIG. 17 shows that the battery pack 120 is removeable from the chest device 110 and makes electrical connection with the chest device 110 via a connector system including connectors 115 and 125, discussed in more detail below.

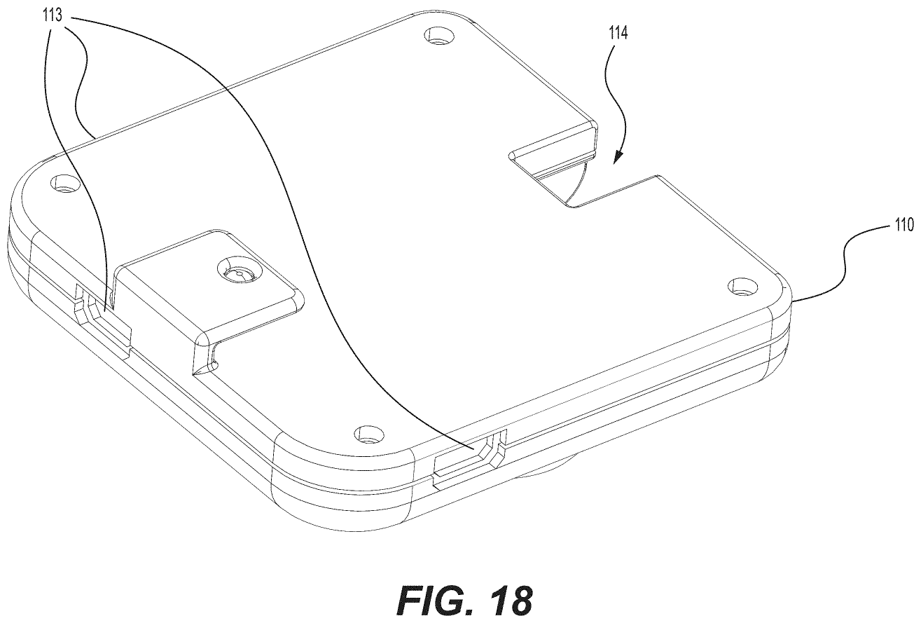

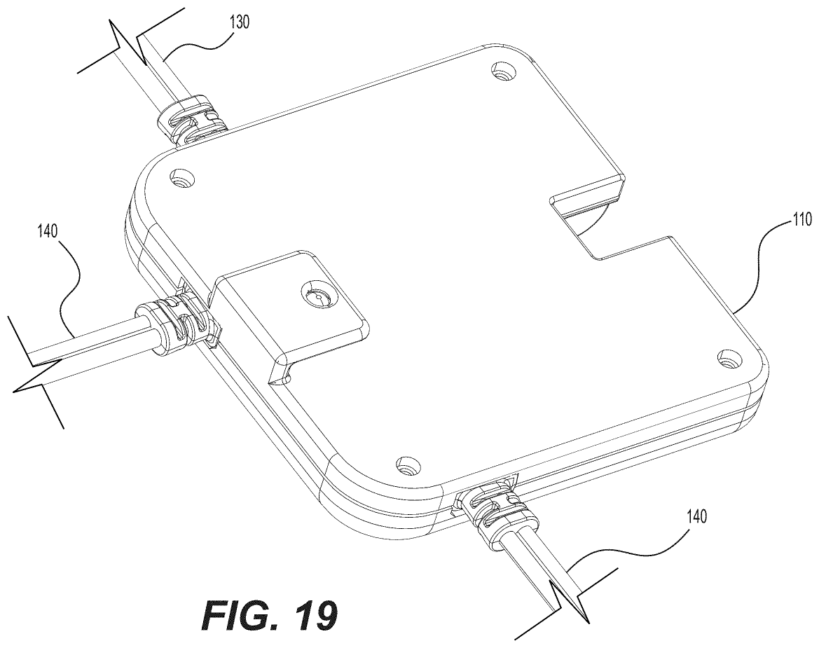

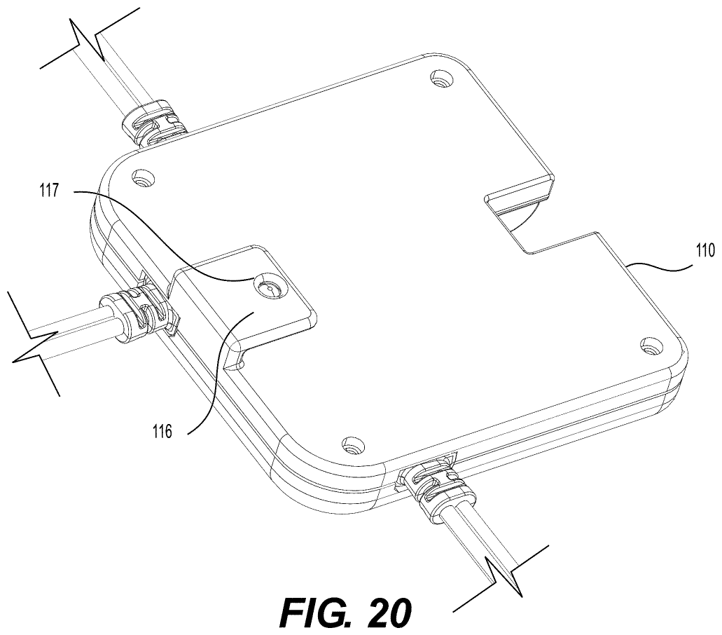

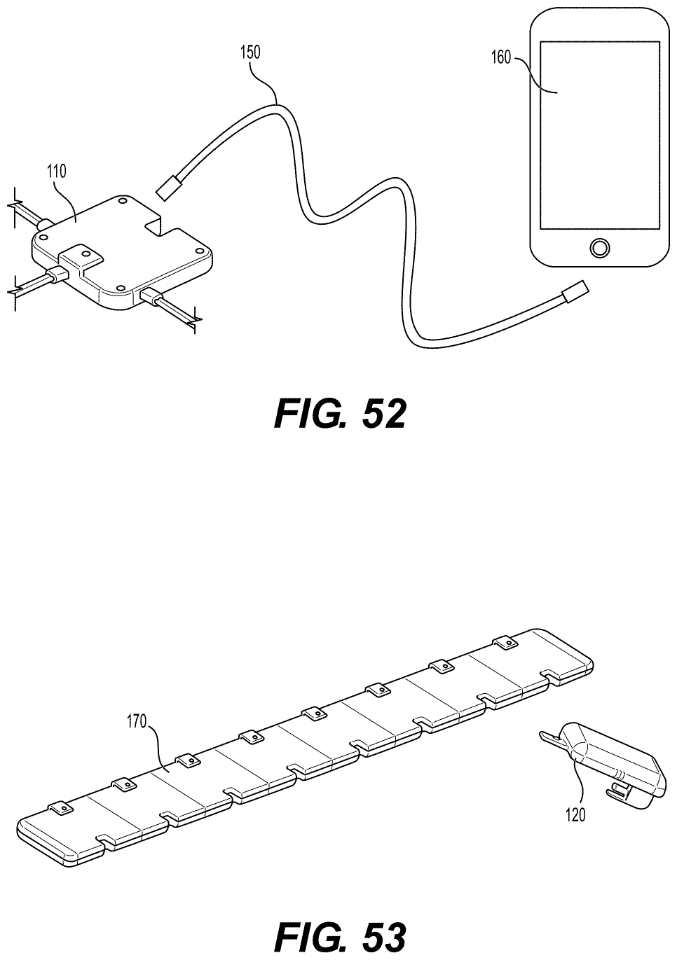

External features of the chest device 110 are described with respect to FIGS. 18-20. FIG. 18 shows that the chest device 110 can be substantially rectangularly shaped with rounded corners and can be made from a clam-shell type construction in which a top housing is attached to a bottom housing, the interior of which houses electronic circuitry to perform patient monitoring and communication operations. FIG. 18 shows that the chest device 110 can include openings 113 in which through the cables 140 for the leads and electrodes 141 and the cable 130 of the pulse ox sensor 131 can be attached. The openings 113 can all have identical shapes or can have different shapes. If a cable or component is not needed, then a plug (not shown) can be inserted in the corresponding opening 113. For example, if the pulse ox sensor 131 is not needed, then a plug can be inserted into the opening 113 that corresponds to the pulse ox sensor 131. The plug can be a permanent plug inserted during a manufacturing or assembly process of the chest device 110, or can a removable plug that is able to be inserted or removed as needed. For example, a customized chest device 110 with a customized cable combination can be made by replacing an unwanted cable with a plug to close the opening 113 during the manufacturing or assembly process. If the openings 113 have identical shapes, then different shaped plugs do not have to be prepared in the manufacturing or assembly process. FIG. 18 also shows that the chest device 110 can also include a cut out or notch 114 as an alignment feature where the battery pack 120 connects to the chest device 110. FIG. 19 is a view of the chest device 110 in which the pulse ox cable 130 and the cables 140 are attached through the openings 113. FIG. 20 shows that the chest device 110 can include an L-shaped arm 116 with a hole 117 that are provided to support, lock, and retain the battery pack 120, as further described below. As shown in FIG. 52, software on the chest device 110 can be updated using an updating cable 150 connected to, for example, a mobile device 160. However, other computing devices may be to update the chest device 110. In addition, or as an alternative to using the updating cable 150, the software of the chest device 110 can be updated wirelessly.

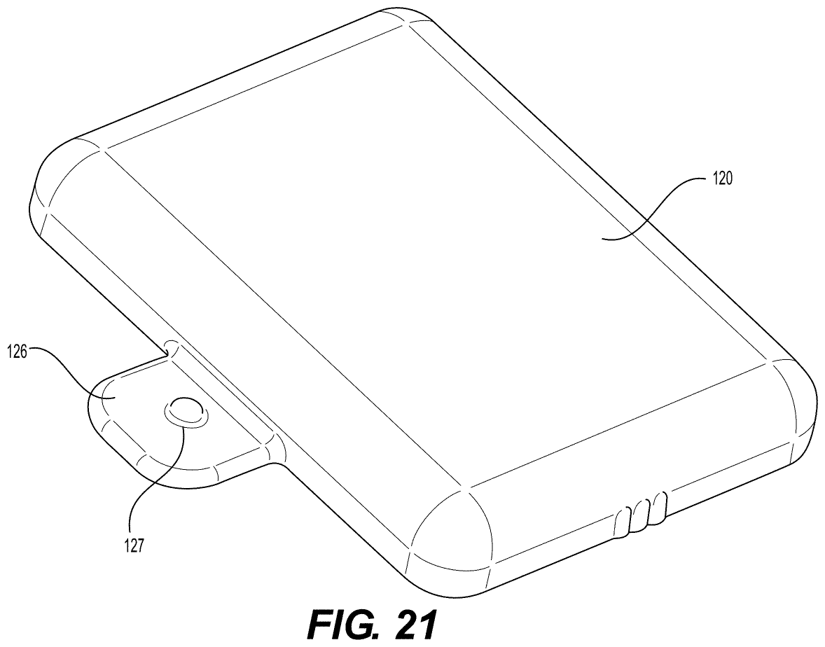

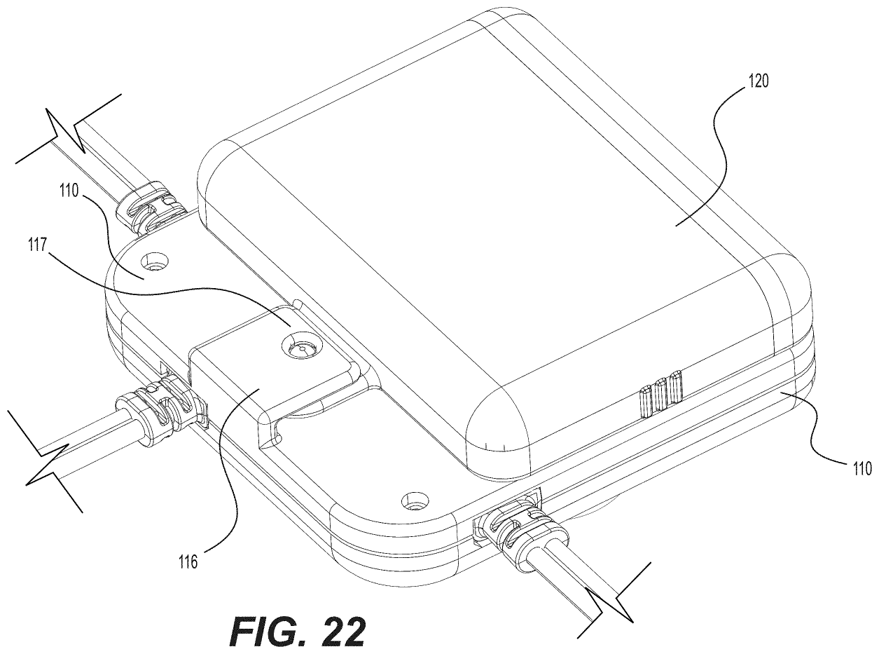

As shown in FIG. 21, the battery pack 120 can be substantially rectangular with rounded corners and include a tab 126 extended from one side of the battery pack 120. The battery pack 120 can include either replaceable batteries or a rechargeable battery in a plastic housing. If the battery pack 120 includes a rechargeable battery, then the battery pack 120 can be charged using a charging station 170, as shown, for example, in FIG. 53. As shown, the tab 126 can include a protrusion or detent 127 that positions and holds the battery pack 120 in relation to the chest device 110. FIG. 22 shows the battery pack 120 in place and engaged with the chest device 110 where the protrusion 127 on the tab 126 of the battery pack 120 is fitted into the hole 117 in the L-shaped arm 116 of the chest device 110 to define a lock structure. The lock structure of the protrusion 127 in the hole 117 helps to properly orient the battery pack 120 with relation to the chest device 110. The lock structure also secures the battery pack 120 in place to the chest device 110 such that more force is required to separate the battery pack 120 from the chest device 110 than the force with which the protrusion 127 is retained in the hole 117. Accordingly, unintentional detachment of the battery pack 120 from the chest device 110 can be prevented, due to the amount of force that must be applied in the proper direction while holding both devices to disengage and remove the battery pack 120. The lock structure also provides a haptic cue, for example, a snap or click feeling to the user, when the protrusion 127 engages or disengages the hole 117.

The chest device 110 does not need to be disconnected or discarded to change or modify the battery pack 120. With a removable battery pack 120, the chest device 110 can remain attached to the patient and does not need to be removed from the electrodes. If the chest device 110 is removed from electrodes, it may be difficult to reattach the chest device 110 to the same electrodes when the electrodes are provided on the patient's skin. Reattaching the chest device 110 to the same electrodes when the electrodes are on the patient's skin includes perfectly or nearly perfectly aligning the snaps 111 of the chest device 110 with snaps of the electrodes and then applying enough pressure to cause the snaps 111 of the chest device 110 to mate with the snaps of the electrodes. In this case, a care giver would need to apply new electrodes to the chest device 110 and then apply the chest device 110 to the patient. If rechargeable, the battery pack 120 when depleted can be replaced with a charged battery pack while the chest device 110 remains attached to the patient. If the battery charge of the battery pack 120 reduces too much or the batteries deteriorate after many times of charging and discharging, a user can simply replace the old battery pack 120 with a new one, without having to replace the chest device 110 that is relatively more expensive than the battery pack 120 because of the circuitry included in the chest device 110.

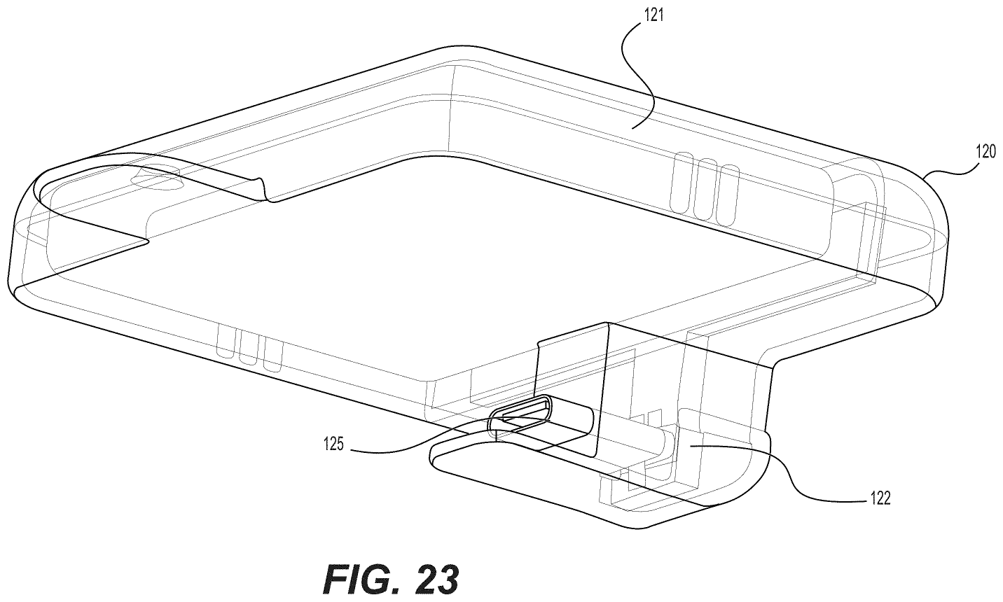

FIG. 23 is a view of the battery pack 120 with the housing shown as transparent. As shown, the battery pack 120 can include a battery 121, a PCB 122, and a connector 125. The PCB 122 routes power and electrical signal interconnections between the battery 121 and the connector 125. The PCB 122 can be rigid or flexible and can include circuitry components. Although the circuitry components can include circuitry components that are not related to battery functions (e.g., functions related to the chest device 110), the battery pack 120 preferably only includes circuitry components related to battery functions (e.g., charging, battery status, etc.). Optionally, the power and electrical signals can be routed by discrete wires or another suitable mechanism. According to the arrangement shown in FIGS. 22 and 23, one of ordinary skill in the art would fully appreciate that the battery pack 120 can change without affecting the chest device 110. That is, the battery pack 120 can vary in thickness to accommodate a different battery that can have a longer life or be made with a different battery technology without having to redesign or reconfigure the chest device 110. As shown in FIG. 22, only one of the top surface, bottom surface, left surface, right surface, front surface, and back surface of the battery pack 120 faces the chest device 110. Accordingly, the battery pack 120 can be implemented with different sizes and different configurations. However, more than one of the top surface, bottom surface, left surface, right surface, front surface, and back surface of the battery pack 120 can face the chest device 110.

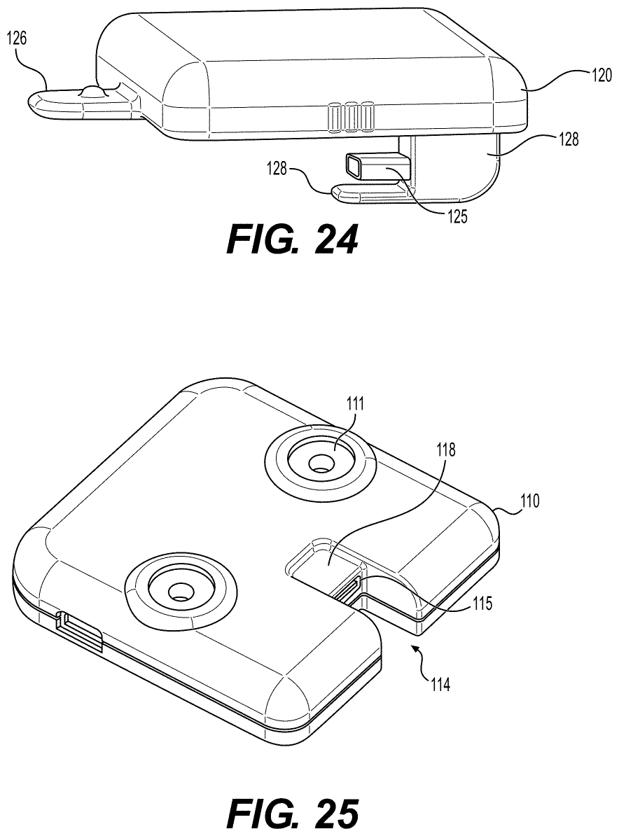

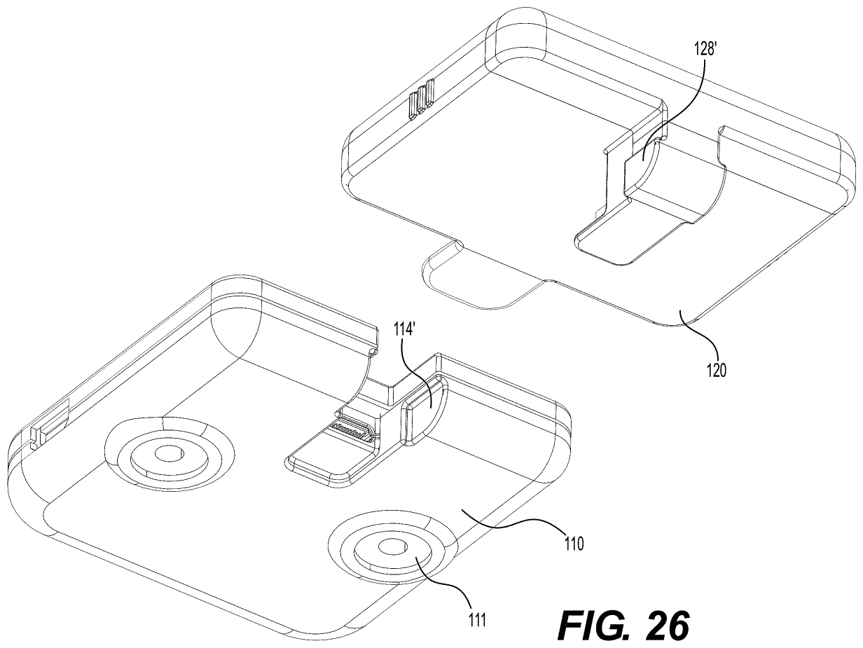

FIGS. 24-26 show connection features of the battery pack 120 and the chest device 110. FIGS. 24 and 26 show that the battery pack 120 can include a beam 128 that projects away from the bottom of the battery pack 120. The beam 128 is a structural feature that retains the connector 125 and encases the interconnection mechanism between the battery 121 and the connector 125.

The connector 125 can be a USB-C, mini-USB, micro-USB, or any other suitable format. As shown in FIGS. 24 and 26, the connector 125 can protrude from the beam 128 in a direction toward the tab 126. The beam 128 can also include a flange 129 located below the connector 125 that also protrudes from the beam 128 in the direction toward the tab 126 and extends further than the connector 125. The flange 129 can have a generally flat shape and protect the connector 125 from physical damage or inadvertent electrical contact.

FIG. 25 shows the chest device 110 with the cut out 114, a connector 115 recessed in the housing of the chest device 110, and a groove 118 located in the cut out 114. FIG. 26 shows the battery pack 120 positioned relative to the chest device 110. When the battery pack 120 and the chest device 110 are joined as shown in FIG. 22, the features of the cut out 114 and the beam 128 provide a key mechanism that properly orients the connector 125 of the battery pack 120 and the connector 115 of the chest device 110 for mating. That is, the inside walls of L-shaped arm 116, the cut out 114, and the groove 118 of the chest device 110 restrict relative movement of the tab 126, the beam 128, and the flange 129 of the battery pack 120 to one axis during engagement and disengagement of the connector 125 of the battery pack 120 and the connector 115 of the chest device 110 so that the connector 125 of the battery pack 120 and the connector 115 of the chest device 110 are properly aligned. The movement axis is parallel to the connector insertion direction of the battery pack 120, so no irregular force is applied to the connector 125 of the battery pack 120 and the connector 115 of the chest device 110 while inserting or removing the battery pack 120. This prevents stress on, damage to, or breakage of the connector 125 of the battery pack 120 and the connector 115 of the chest device 110. Different arrangements of the L-shaped arm 116, the cut out 114, and the groove 118 of the chest device 110 and of the tab 126, the beam 128, and the flange 129 of the battery pack 120 can be used. For example, the battery pack 120 can include a cut out and a groove, and the chest device 110 can include a beam and a flange. It is also possible to use other guiding structures that restrict relative movement of the battery pack 120 with respect to the chest device 110 to one axis during engagement and disengagement of the battery pack 120 and the chest device 110 and/or that ensure no irregular force is applied to the connector 125 of the battery pack 120 and the connector 115 of the chest device 110 while inserting or removing the battery pack 120.

Additionally, the structural orientation of the cut out 114 and the connector 115 in the chest device 110 and the beam 128 and the connector 125 of the battery pack 120 prevents the user from accidentally inserting another connector and mitigates any risk of electric shock to the patient and of device failure by misuse. The chest device 110 should only engage with the battery pack 120 and, as shown in FIG. 52, with an updating cable 150 that can be connected to, for example, a mobile device 160. The cut out 114 can be arranged to define portions of the chest device 110 that protrude beyond the interface of the connector 115 of the chest device 110 to interfere with any other connector being inserted into the connector 115 of the chest device 110. The width of the cut out 114 and the corresponding width of the beam 128 can be narrow enough to allow insertion of the beam 128 but to interfere with any other connector being inserted into the connector 115 of the chest device 110. It is also possible to use other erroneous insertion prevention structures on the chest device 110 that prevent insertion of an incompatible connector and/or that protect the chest device 110 from being damaged. The flange 129 can protect the Battery of the battery pack 120 from being damaged. The flange 129 can be close enough to the connector 125 of the battery pack 120 to allow the connector 125 of the battery pack 120 to be mated with the connector 115 of the chest device 110 but also to interfere with any non-compatible connector being mated with the connector 125 of the battery pack 120. It is also possible to use other erroneous insertion prevention structures on the battery pack 120 that prevent insertion of an incompatible connector and/or that protect the Battery of the battery pack 120 from being damaged. The battery pack 120 should only engage with the chest device 110 and, as shown in FIG. 53, with a charging station 170. Once fully engaged, the beam 128, the cut out 114, the flange 129, and the groove 118 respectively fit tightly together. Accordingly, the lock structure and the matching geometric arrangement of the battery pack 120 and the chest device 110 ensure a highly reliable mating system.

FIG. 26 shows an alternative mating implementation of the sides walls of the cut out 114 and the beam 128 that is different than that shown in FIGS. 24 and 25. FIG. 26 shows that the side walls of the cut out 114 can include a recess defining a step 114′ that can accept a corresponding thicker portion 128′ of the beam 128. Variations of this feature can be used to ensure different generations of the chest device 110 and the battery pack 120 can only mate with compatible products.

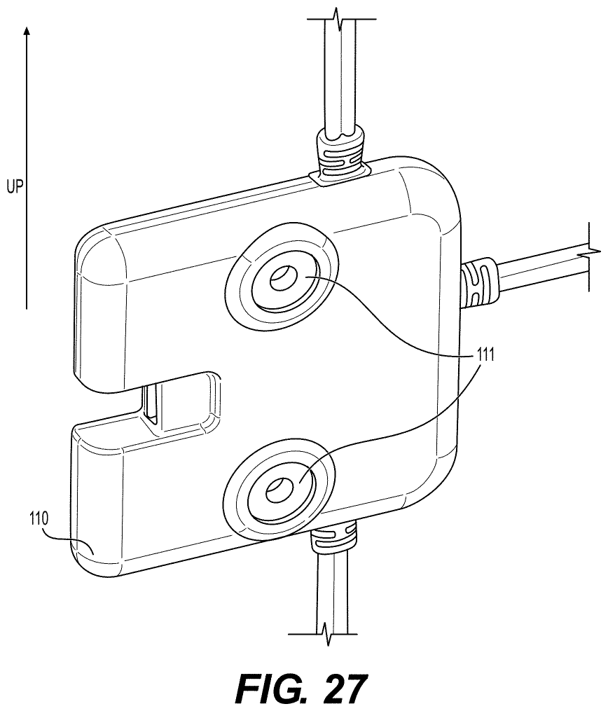

Orientation of the patient monitoring device 100 on the patient is discussed below with respect to FIGS. 27-31B. Unlike the conventional patient-worn monitor device in which the snaps on the adapter are oriented to be horizonal across a patient's chest, the snaps 111 on the chest device 110 according to preferred embodiments of the present invention are oriented vertically across the patient's chest, as shown in FIG. 27. FIG. 27 shows that cables can be attached to the chest device 110 at the top, the bottom, and the right side.

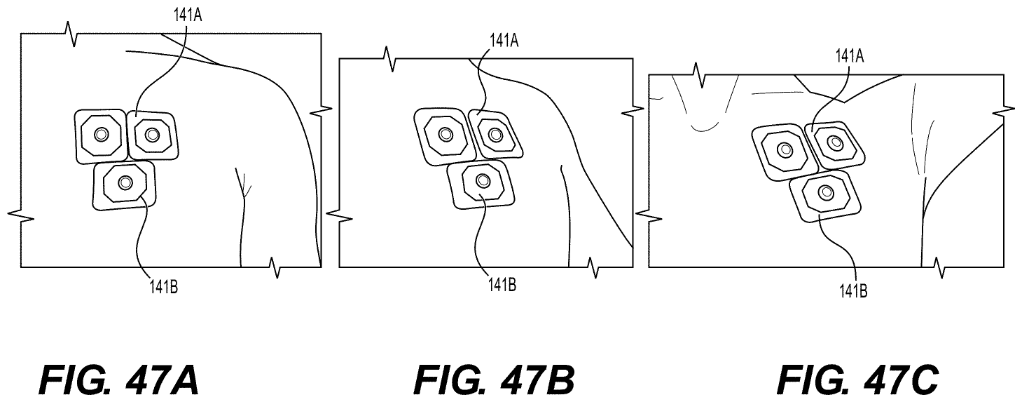

FIG. 47A shows a grouping of skin electrodes on a patient's chest when a patient is at rest, with the top two skin electrodes 141A being directly adjacent to each other in a horizontal direction, and with the bottom skin electrode 141B directly adjacent to the top two skin electrodes 141A in the vertical direction. As shown with the top two skin electrodes 141A in FIGS. 47B and 47C, when a patient moves or changes arm position, a patient's skin in the chest area stretches in the horizontal direction during movement and causes the top two skin electrodes 141A to separate horizontally, which if connected to a conventional adapter can cause the top two skin electrodes 141A to separate and provide stress between corresponding adapter snaps (for example, the snaps 111) and the electrodes 141A and 141B. This added stress can cause the patient-worn device to pop off from the skin electrodes 141A and 141B. However, as shown with the bottom skin electrode 141B with respect to the top two skin electrodes 141A in FIGS. 47B and 47C, the patient's skin in the chest area does not stretch or does not stretch as much in the vertical direction, causing no or little movement in the vertical direction between the bottom skin electrode 141B with respect to the top two skin electrodes 141A. That is, when a patient moves, the patient's pectoral skin tends to stretch laterally (or horizontally) rather than longitudinally (or vertically), which reduces the chances of a chest device (for example, the chest device 110) from popping off of the snaps (for example, the snaps 111) are located longitudinally (or vertically). Therefore, providing the snaps 111 in a vertical orientation on the rear of the chest device 110 eliminates or significantly reduces the problem of stress between adapter snaps 111 and electrodes. The lines of non-extension in which the skin does not move during movement that are demonstrated in FIGS. 47A to 47C are particular to the chest area and could be different in another area of the patient's skin. Thus, a different orientation of the skin electrodes may be provided according to different areas of the patient's skin.

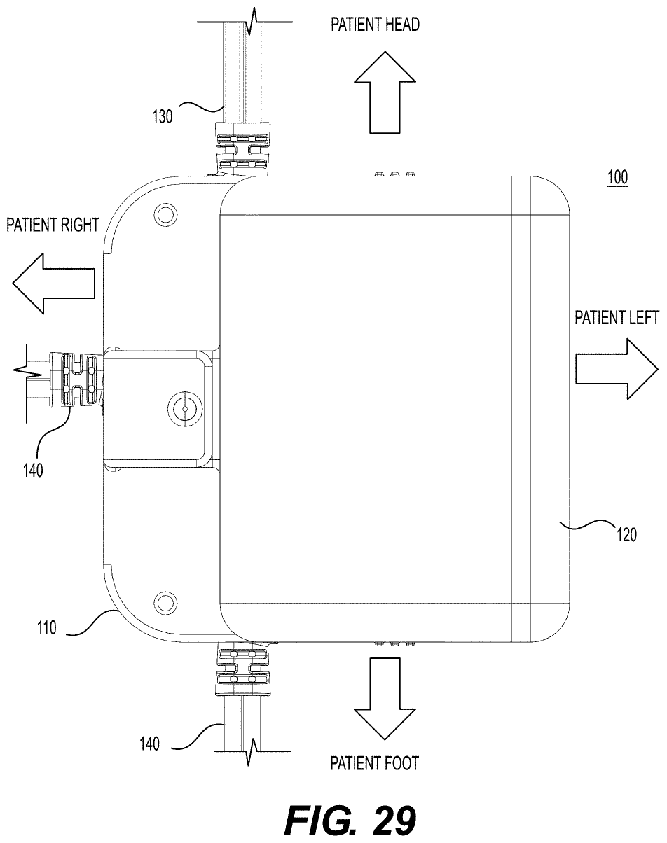

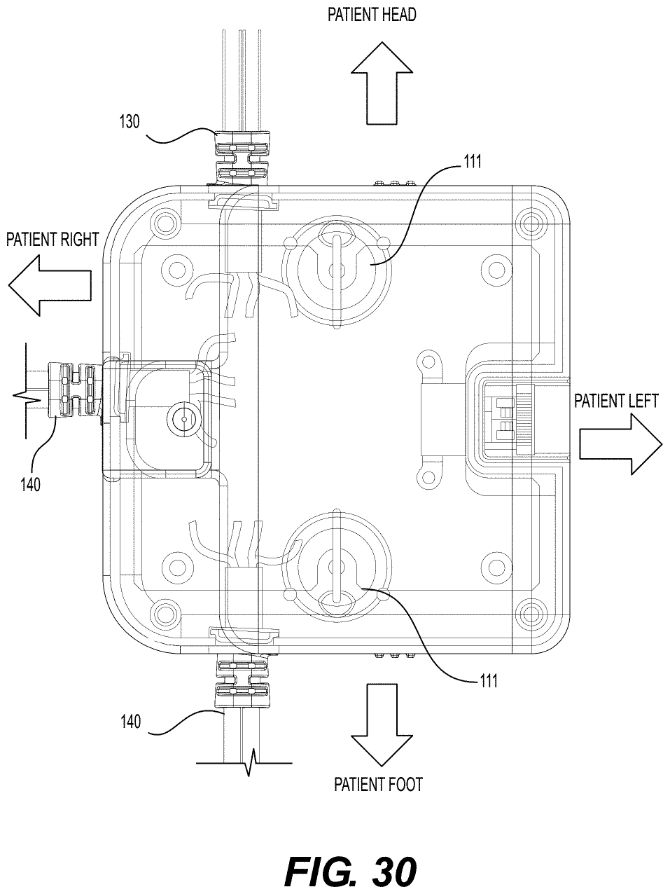

The orientation of the patient monitoring device 100 according to a preferred embodiment of the present invention is shown in FIGS. 28-30. FIG. 28 is an illustration of the patient monitoring device 100, ECG leads and electrodes 141, respiration sensing leads and electrodes 142, and a pulse ox sensor 131 attached to a patient. As shown, cables 140 for the ECG leads and electrodes 141 and the respiration sensing leads and electrodes 142 are attached to the patient monitoring device 100 at the bottom and right side, and the pulse-ox cable 130 to the pulse ox sensor 131 is attached to the top of the patient monitoring device 100. This orientation is further shown in FIGS. 29 and 30.

FIGS. 29 and 30 are views of the front of the patient monitoring device 100 as oriented when attached to a patient and indicate the directions toward the patient's head and foot and to the left and right sides of the patient. FIG. 30 is a view with the housings of the battery pack 120 and chest device 110 shown as transparent so that the vertically oriented snaps 111 at the rear of the chest device 110 are visible.

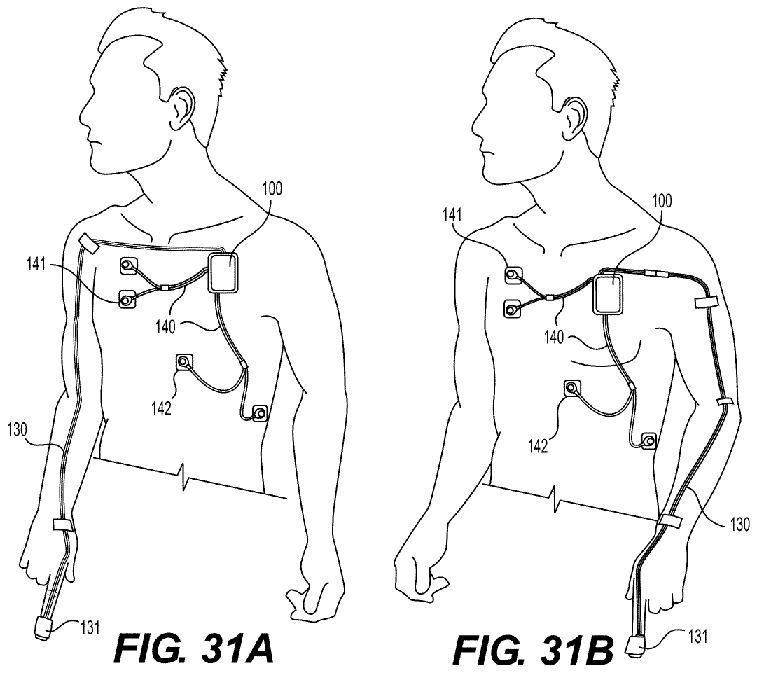

FIGS. 31A and 31B show two implementations of the patient monitoring device 100 attached to a patient. Because the battery pack 120 is attached to the chest device 110 from the patient's left side, the cable 130 for the pulse ox sensor 131 can be attached to the top of the chest device 110, instead of the side of the chest device 110. Because the cable 130 for the pulse ox sensor 131 is attached to the top of the patient monitoring device 100, the pulse-ox cable 130 can easily be routed to either side of the patient. FIG. 31A shows the pulse ox sensor 131 routed to the left and attached to the patient's right hand. FIG. 31B shows the pulse ox sensor 131 routed to the right and attached to the patient's left hand.

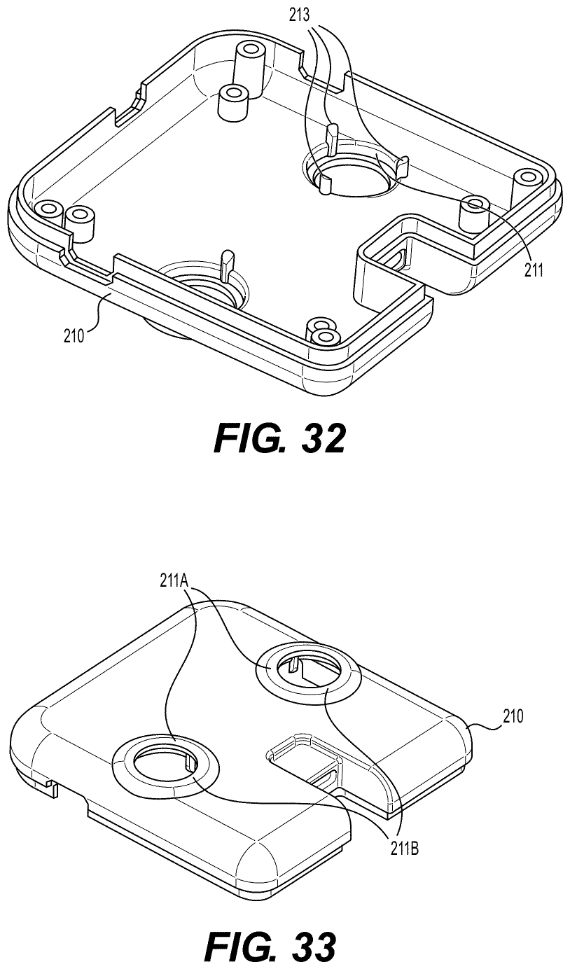

FIGS. 32 and 33 are views of an example of a bottom housing 210 of the chest device 110. FIG. 32 is a view of the interior of the bottom housing 210 showing features used to locate and mount the snaps 111. FIG. 32 shows circular cavities 211 with a recessed step and a circular opening in the middle. The cavities 211 are used to position the snaps 111 within the bottom housing 210, but other structures could be used to position the snaps 111 within the bottom housing 210. FIG. 32 further shows three ribs 213 protruding from the inside surface of the bottom housing 210 and located directly adjacent to each cavity 211 and along an outer perimeter of the cavity 211. The ribs 213 assist in locating and retaining the snaps 111 in the desired position during assembly until a potting compound 219 is cured, as will be described below. Alternatively, a different number of ribs 213 can be used. As shown in the rear view of FIG. 33, the recess of the cavities 211 creates protrusions 211A on the outside of the bottom housing 210. The protrusions 211A are raised from the main body of the rear surface of the bottom housing 210 and terminate in flat surfaces 211B. The flat surfaces 211B create space between the main body of the rear surface of the bottom housing 210 and the skin-mounted electrodes 141 when attached to a patient. This space created allows the chest device 110 to slightly tilt with respect to the skin-mounted electrodes 141 so that the chest device 110 has freedom to follow the skin movement of the patient. Accordingly, the bottom housing 210 need not have a rubber soft boot around the snaps 111, as is required by conventional designs, because the structure of the bottom housing 210 allows for movement between the chest device 110 and the skin-mounted electrodes 141 similar to movement that is provided by conventional soft boots.

Soft boots are made by over-molding of rubber-like material onto a plastic enclosure. The over-molding of the rubber-like material is complicated and costly. Soft boots can be eliminated if vertically-arranged electrodes 141 are used because the electrodes 141 do not need to move relative to each other. Accordingly, the complicated and costly process of manufacturing soft boots is unnecessary and eliminated by preferred embodiments of the present invention. Eliminating the soft boots simplifies the design of the chest device 110 and reduces the size and weight of the chest device 110, which is beneficial to the patient. Cavities 211 on the chest device 110 are made of strong enough material that they can be big enough to fit plastic electrode snaps and still hold the device snaps 111 securely with no pop-off and pull-out problems.

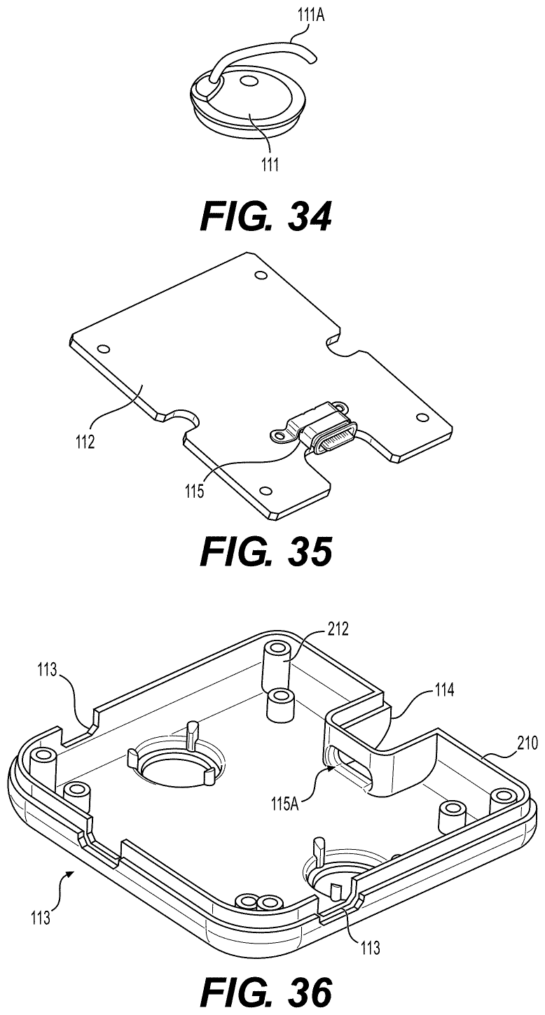

FIGS. 34-44 are used to describe an example of an assembly process of the chest device 110. FIG. 34 shows that a wire 111A can be soldered to each snap 111. FIG. 35 shows that a PCB 112 can be provided that includes the connector 115 and mounted circuitry components (not shown). The PCB 112 can be rigid and made from any suitable material. Any suitable substrate can be used instead of the PCB 112.

FIG. 36 shows that the bottom housing 210 can be provided with a clam-shell type construction. The bottom housing 210 can be made of a plastic or any suitable material and can be made by casting, molding, injection molding, machining, or any suitable method or combination thereof. The bottom housing 210 can include the openings 113, cut out 114, ribs 213, and cavities 211 as previously described. The bottom housing 210 can also include a connector opening 115A to access the connector 115 and bosses 212 to mount the PCB 112 and to attach a top housing 310.

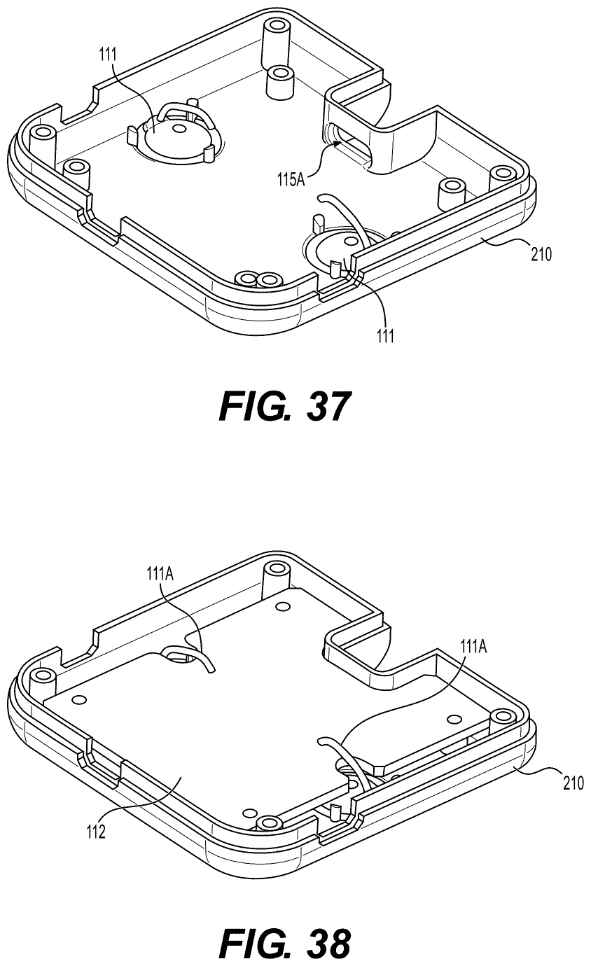

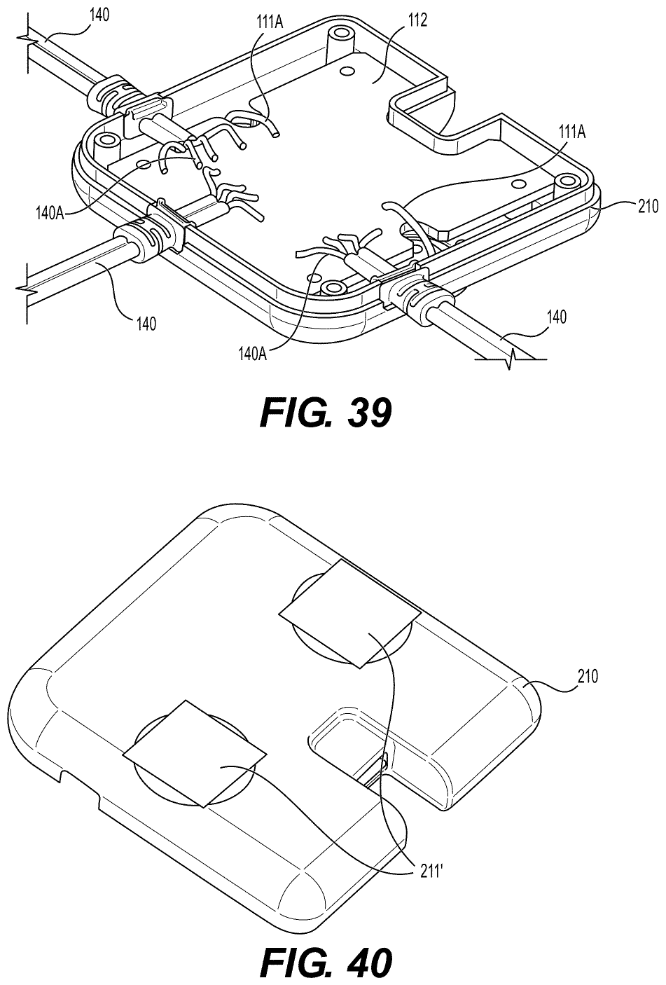

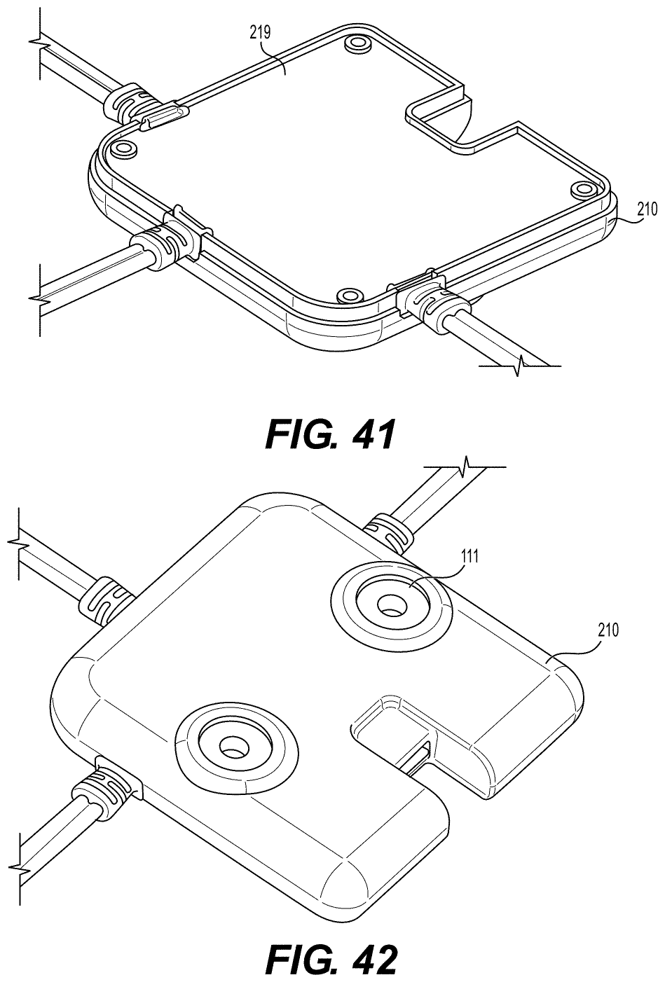

FIG. 37 shows two snaps 111 with wires 111A positioned in respective cavities 211 of the bottom housing 210. FIG. 38 shows the PCB 112 positioned in the bottom housing 210 where the connector 115 is on the rear side (not shown). The PCB 112 can be fixed to the bottom housing 210 by press-fit, by using fasteners or an adhesive, or by heat-staking. When the PCB 112 is fixed to the bottom housing 210 there is a space between the PCB 112 and the snaps 111. FIG. 39 shows that wiring 111A for the snaps 111 and wiring 140A for the cables 140 can be soldered or connected to the PCB 112. When filling the bottom housing 210 with the potting compound 219 to cover the snaps 111 and the PCB 112, an air void can remain under PCB 112, which can cause the chest device 110 to fail during defibrillation. The PCB 112 can include small holes that act as air vents, which decreases the risk of air voids being formed by the potting compound 219. In addition to or as an alternate to using a PCB 112 with small holes, at least a portion of the bottom housing 210 and/or top housing 310 can be transparent so that any air voids formed by the potting compound 219 under the PCB 112 are visible after the assembly of the chest device 110 is assembled. FIG. 40 shows that the cavities 211 for the snaps 111 on the rear surface of the bottom housing 210 can be covered by tape 211′ to prevent leakage of potting compound 219 shown in FIG. 41. FIG. 41 shows that the interior of the bottom housing 210 can be filled with dielectric potting compound 219 that is cured to cover and encase the PCB 112, the snaps 111, soldered wires 111A and 140A, and circuit components mounted on the PCB 112. The potting compound 219 can be an epoxy resin, silicone, or any other suitable material. The potting compound 219 can have a relative permittivity of less than four (4). FIG. 42 shows that the tape 211′ can be removed from the rear surface of the bottom housing 210.

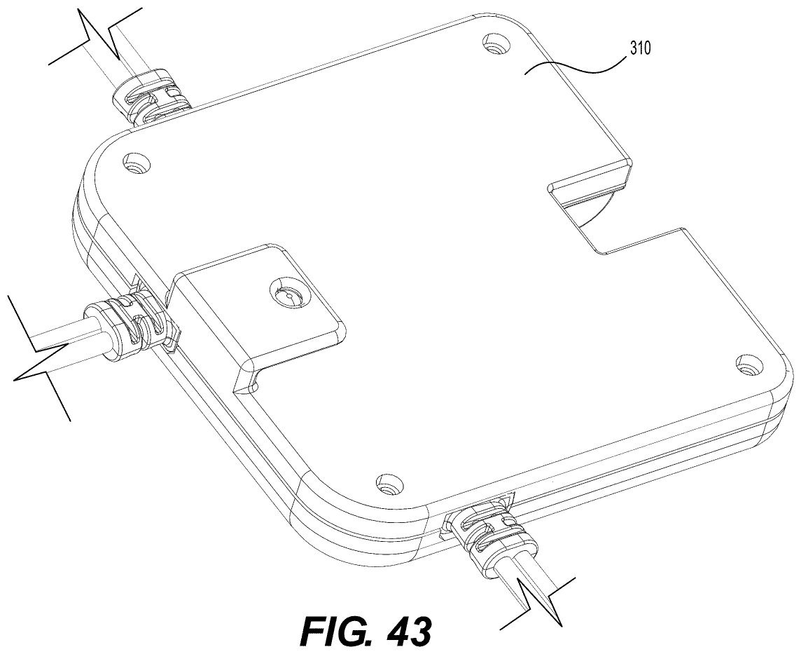

FIG. 43 shows that a top housing 310 can be attached to the bottom housing 210 to complete assembly of the chest device 110. The top housing 310 can be made of a plastic or any suitable material and can be made by casting, molding, injection molding, machining, or any suitable method or combination thereof. The top housing 310 can be attached to the bottom housing 210 by press-fit as being snapped into place or using fasteners, an adhesive, or any other suitable method.

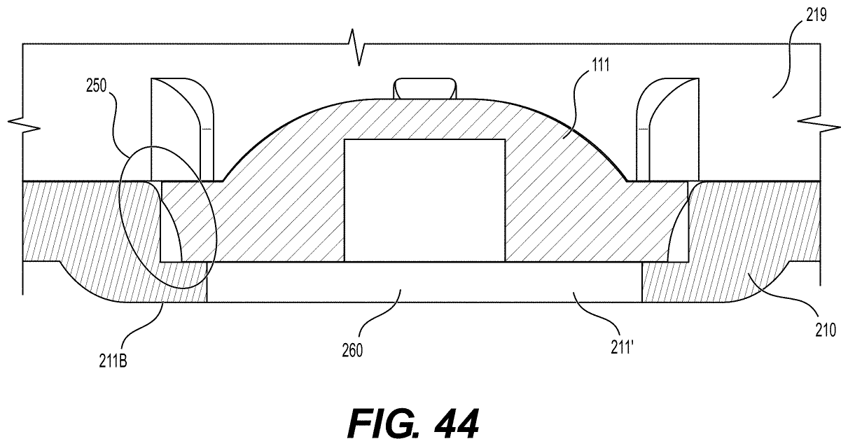

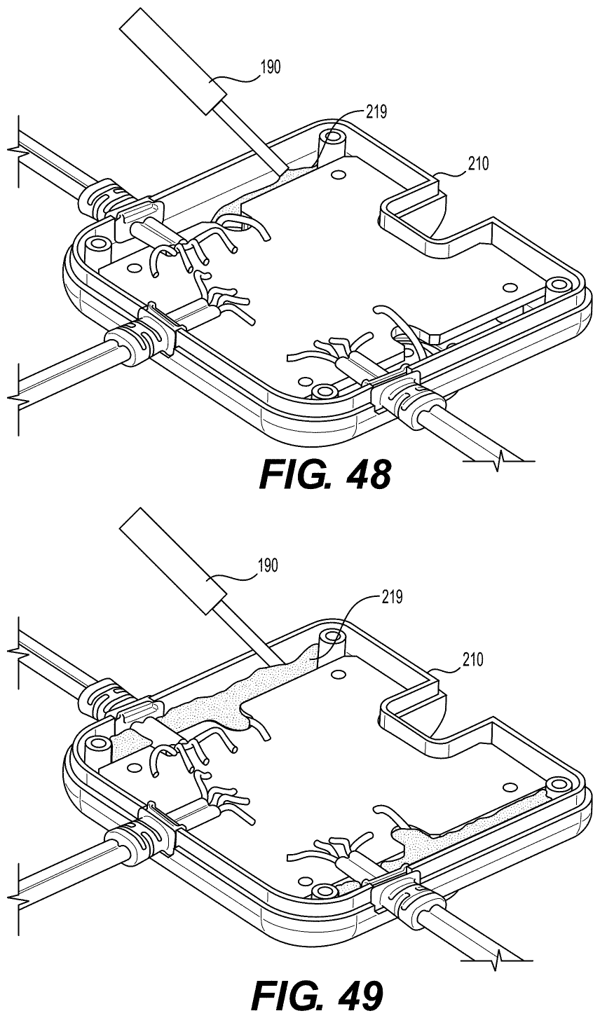

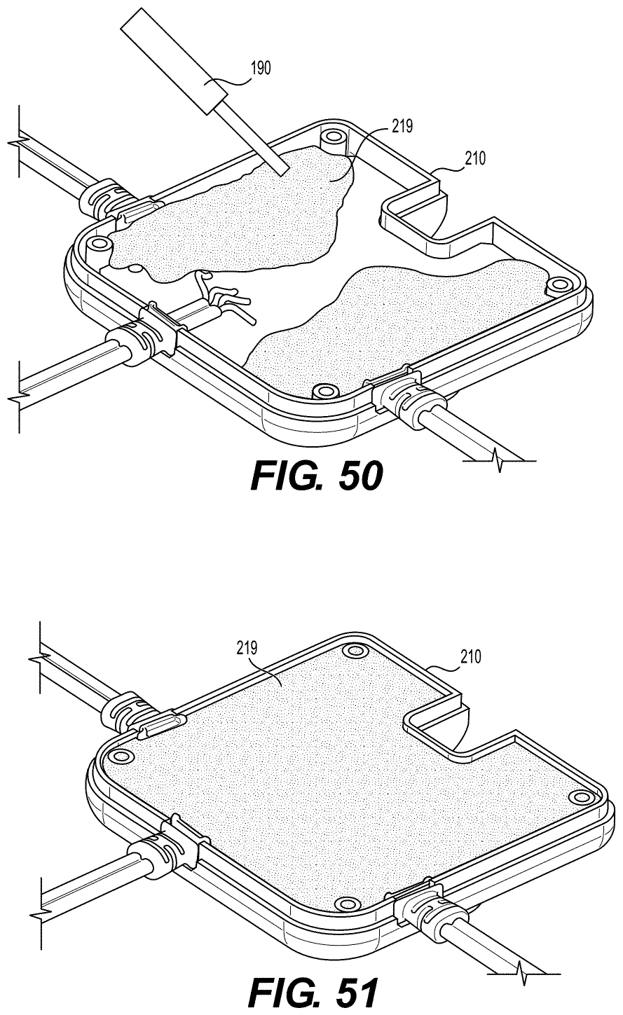

FIG. 44 is a section view of the snap area during the process of applying the potting compound 219. In the assembly process shown in FIGS. 48-51, the potting compound 219 in fluid form is applied by an applicator 190 to flow under the PCB 112, cover the PCB 112, and cover the snaps 111 inside the cavities 211. There can be a small gap between each snap 111 and the bottom housing 210 if the bottom housing 210 is not made of rubber or soft plastic, making a perfect seal difficult or impossible. Before the potting compound 219 is cured and solidified, the potting compound 219 can leak through a small gap between contact surfaces 250 of the snap 111 and the bottom housing 210. If this happens, the potting compound 219 can adhere to the outer surface of the snap 111, which can lead to the product being defective by preventing an electrical connection between the snaps 111 and the skin-mounted electrodes 141. To prevent this failure, the tape 211′ is applied to the Flat Surface of the bottom housing 210 to seal the cavities 211 before the potting process. The resulting enclosed air 260 is compressed by the flowing potting compound 219, and the air pressure prevents the potting compound 219 from flowing into the cavities 211 and causing the snaps 111 to be defective. After the potting compound 219 is cured, the tape 211′ is removed. Therefore, the bottom housing 210 need not be made of rubber-like material because a perfect seal between the snaps 111 and the bottom housing 210 is not required during assembly. As explained above, the bottom housing 210 can be made transparent so that inspection of the potting process can be performed under the PCB 112.

Additionally, potting the internal electrical components of the bottom housing 210 saves space needed in the conventional design to separate the snaps 111 from sources of possible electrical arcing caused by, for example, defibrillation. Filling space between possible arcing sources with dielectric material, such as potting compound 219, significantly reduces or prevents the risk of electrical arcing, allows for more dense component packaging, and reduces the overall size of the chest device 110 compared to conventional adapter-sensor assemblies. Because the potting compound 219 encapsulates the PCB 112 and the electrical components on the PCB 112, as well as the snaps 111 and wires 111A, a dense and compact electrical assembly is provided without concern of failure during defibrillation. As a result, a smaller and lighter weight design provided by the chest device 110 can reduce an uncomfortable burden on the patient.

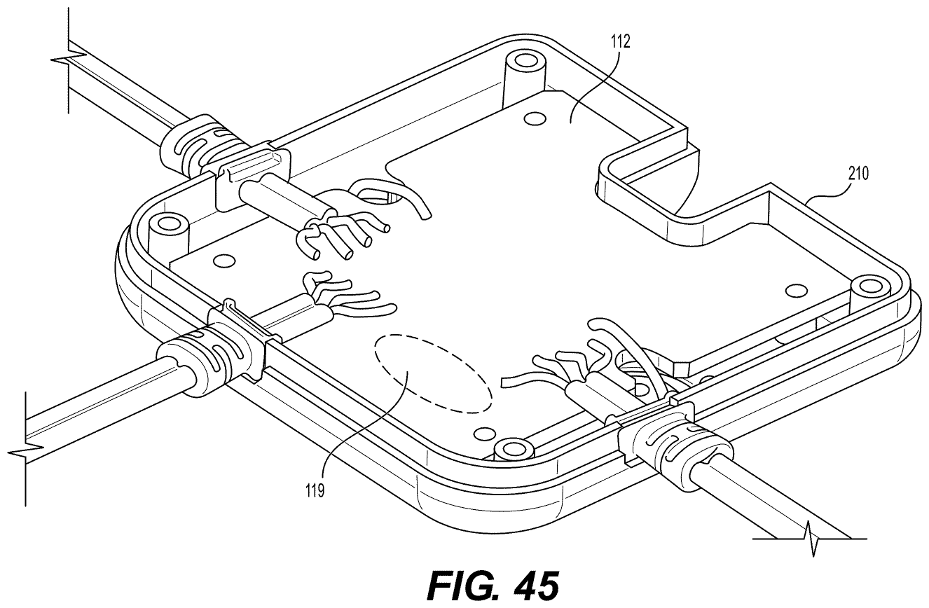

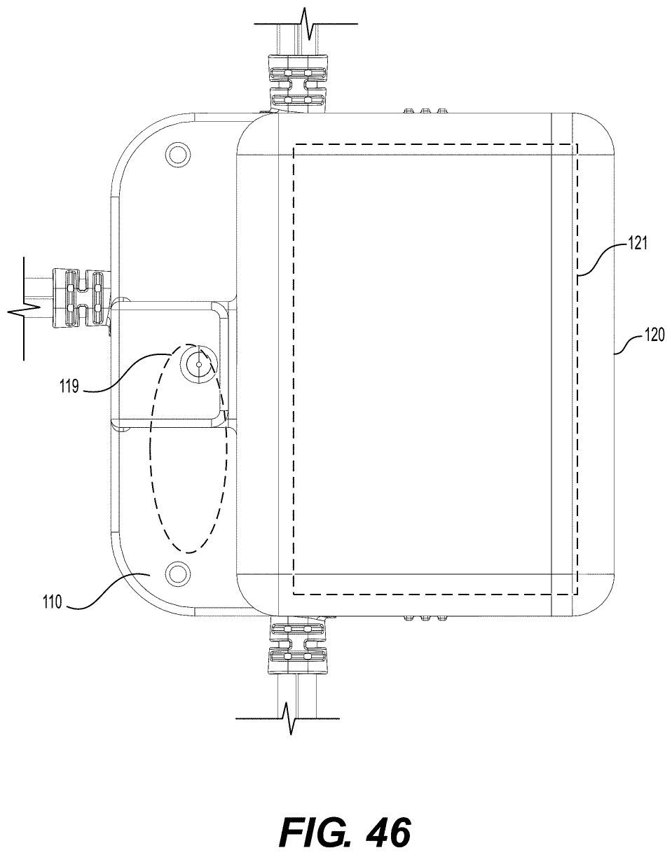

FIGS. 45 and 46 show an antenna implementation according to a preferred embodiment of the present invention. To provide reliable connectivity between the patient monitoring device 100 and a remote monitoring system, an antenna on the PCB 112 should be able to freely send and receive wireless communications. Accordingly, an antenna on the PCB 112 should not be covered by a battery as the battery could provide a shielding effect and prevent reliable operation of the antenna. In a preferred embodiment of the present invention, the battery 121 in the battery pack 120 does not completely overlap the PCB 112 in plan view, i.e., in a direction perpendicular to a major surface of the PCB 112 or in a direction towards the patient when the chest device 110 is attached to a patient. For example, FIG. 45 shows an antenna location 119, approximated by a dashed oval, on the PCB 112. FIG. 46 shows the patient monitoring device 100 with the battery pack 120 engaged with the chest device 110. The dotted box approximates the location of the battery 121 that is offset to one side of the chest device 110 and does not entirely overlap with the underlying PCB 112 and the antenna location 119, as shown by the dashed oval. Because the antenna can be provided in antenna location 119, which is an area of the PCB 112 that is not covered by the battery 121, radio waves to and from the antenna are not blocked by the battery 121. Furthermore, as shown in FIG. 45, the antenna location 119 also does not overlap any cable wiring 140A, in plan view, i.e., in a direction perpendicular to a major surface of the PCB 112 or in a direction towards the patient when the chest device 110 is attached to a patient, that could interfere with wireless communication.

The antenna on the PCB 112 is preferably not covered with the potting compound 219 or any other material, in order to reproducibly provide good impedance matching during the assembly process of the chest device 110. For example, a wall feature (not shown) could be adhered to the PCB 112 around the antenna before applying the potting compound 219 to keep an open space around the antenna. However, if the antenna on the PCB 112 is covered by the potting compound 219, the potting compound 219 preferably has a low permittivity (dielectric constant) to maximize antenna efficiency and to provide reproducible good impedance matching during the assembly process of the chest device 110.

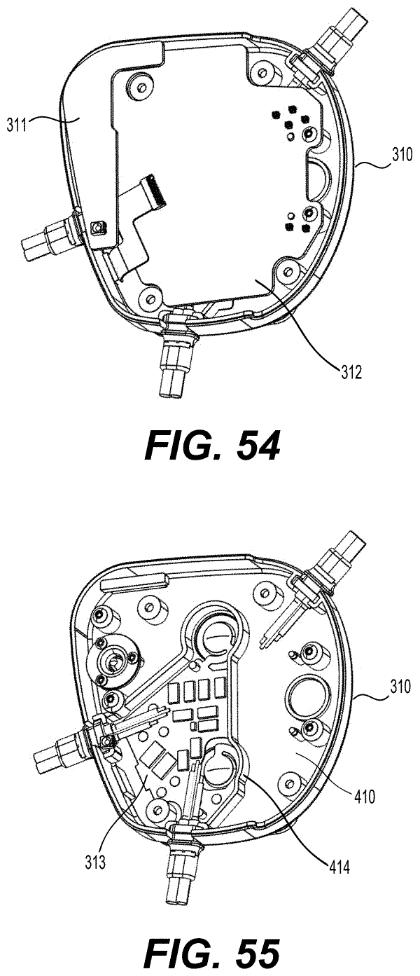

FIGS. 54-57 show a modified chest device 310 according to a preferred embodiment of the present invention. The modified chest device 310 is similar to chest device 110, except that the modified chest device 310 can include three separate PCBs 311, 312, 313.

As shown in FIGS. 54 and 57, the modified chest device 310 can include a first PCB 311 that is located to not overlap a battery 321 included in a battery pack 320, in plan view, i.e., in a direction perpendicular to a major surface of the PCB 311 or in a direction towards the patient when the chest device 310 is attached to a patient. Accordingly, the first PCB 311 can include circuitry for wireless communication and the like. As further shown in FIG. 54, the modified chest device 310 can include a second PCB 312, which can be similar to the PCB 112 of the chest device 110.

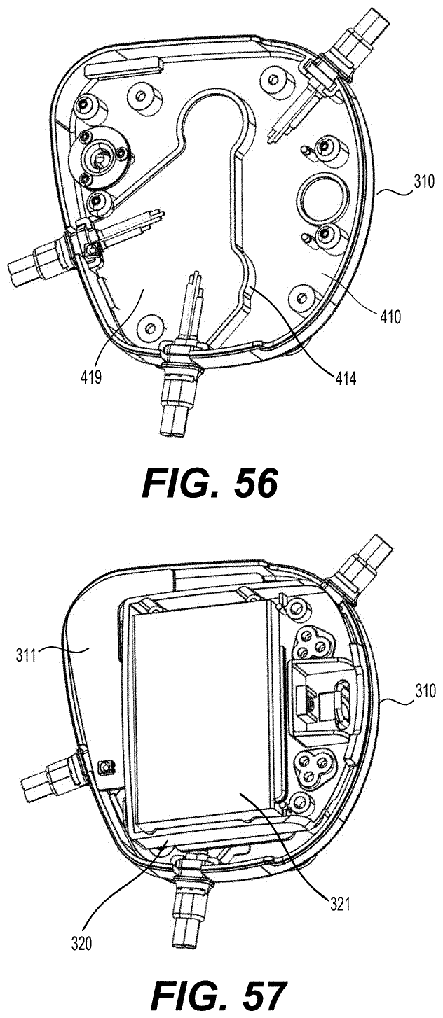

As shown in FIG. 55, the modified chest device 310 can additionally include a third PCB 313 that is provided below the second PCB 312 and on a bottom housing 410 of the modified chest device 310. The bottom housing 410 can include a retaining wall 414 that extends around the perimeter of the third PCB 313. As shown in FIG. 56, the third PCB 313 can be covered by a potting compound 419, similar to the potting compound 219 included in the chest device 110. The retaining wall 414 of the bottom housing 410 can limit the flow of the potting compound 419 to an area surrounding the third PCB 313, such that the potting compound 419 is prevented from flowing to other portions of the bottom housing 410. Accordingly, in the modified chest device 310, potting can be applied only to circuit components included on the third PCB 313, with potting not being applied to circuit components included on the first PCB 311 and the second PCB 312. Thus, by only potting the circuit components which have been included on the third PCB 313, for example, analog circuit components, a weight of the potting compound 419 included in the modified chest device 310 can be reduced compared to a weight of the potting compound 219 included in the chest device 110. That is, the modified chest device 310 can provide an overall reduction in weight by only potting specific circuit components included on a separate PCB.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1: A patient-monitoring device comprising:
a housing including a first opening;
a snap located in the first opening to connect to an electrode through the first opening; and
a dielectric potting compound covering the snap.

2: The patient-monitoring device of claim 1, further comprising a flat surface surrounding the first opening.

3: The patient-monitoring device of claim 2, further comprising a protrusion that defines the flat surface.

4: The patient-monitoring device of claim 1, wherein the housing further includes a plurality of ribs spaced around the first opening.

5: The patient-monitoring device of claim 1, further comprising a second opening, wherein
the first opening and the second opening are aligned to be vertical or substantially vertical when the patient-monitoring device is mounted to a patient.

6: The patient-monitoring device of claim 1, further comprising:
a first cavity that defines the first opening; and
a second cavity that defines the second opening.

7: The patient-monitoring device of claim 1, further comprising a battery pack that is attached to and detached from the housing, wherein
the battery pack includes a battery to power the patient-monitoring device.

8: The patient-monitoring device of claim 7, wherein
the housing includes a first connector, and
the housing aligns and restricts a direction in which the battery pack is attached to and detached from the first connector.

9: The patient-monitoring device of claim 7, wherein
the housing includes a first lock, and
the battery pack includes a second lock that communicates with the first lock to lock the battery pack to the housing.

10: The patient-monitoring device of claim 7, further comprising an antenna within a portion of the housing that is not overlapped, in plan view, with the battery pack when the battery pack is attached to the housing.

11: The patient-monitoring device of claim 10, wherein the dielectric potting compound does not cover the antenna.

12: The patient-monitoring device of claim 1, wherein a relative permittivity of the dielectric potting compound is less than 4.

13: The patient-monitoring device of claim 1, wherein
the housing further includes an antenna, and
the dielectric potting compound does not cover the antenna.

14: The patient-monitoring device of claim 1, further comprising a printed circuit board that is in the housing.

15: The patient-monitoring device of claim 14, wherein the printed circuit board includes holes.

16: The patient-monitoring device of claim 14, wherein the dielectric potting compound covers the printed circuit board.

17: The patient-monitoring device of claim 16, further comprising a secondary printed circuit board that is in the housing, wherein
the dielectric potting compound does not cover the secondary printed circuit board.

18: The patient-monitoring device of claim 16, wherein
the housing includes a retaining wall that extends around a perimeter of the printed circuit board; and
the dielectric potting compound is within the retaining wall.

19: The patient-monitoring device of claim 1, wherein at least a portion of the housing is transparent.

20: The patient-monitoring device of claim 7, wherein at least one of a top surface, a bottom surface, a left surface, a right surface, a front surface, and a back surface of the battery pack does not oppose the housing.

21: The patient-monitoring device of claim 1, wherein the housing includes openings that include an identical shape and that each receive a cable or plug.
